SEQUENCE LISTING (31 sequences)

SEQ ID NO: 1 - protein, 1088 aa, Gracilariopsis lemaneiformis
MFSTLAFVAPSALGASTFVGAEVRSNVRIHSAFPAVHTATRKTNRLNVSMTALSDKQTATAGSTDNPDGIDYKTYDYVGVWGFSPLSNTNWFAAGSSTPGGITDWTATMNVNFDRIDNPSITVQHPVQVQVTSYNNNSYRVRFNPDGPIRDVTRGPILKQQLDWIRTQELSEGCDPGMTFTSEGFLTFETKDLSVIIYGNFKTRVTRKSDGKVIMENDEVGTASSGNKCRGLMFVDRLYGNAIASVNKNFRNDAVKQEGFYGAGEVNCKYQDTYILERTGIAMTNYNYDNLNYNQWDLRPPHHDGALNPDYYIPMYYAAPWLIVNGCAGTSEQYSYGWFMDNVSQSYMNTGDTTWNSGQEDLAYMGAQYGPFDQHFVYGAGGGMECVVTAFSLLQGKEFENQVLNKRSVMPPKYVFGFFQGVFGTSSLLRAHMPAGENNISVEEIVEGYQNNNFPFEGLAVDVDMQDNLRVFTTKGEFWTANRVGTGGDPNNRSVFEWAHDKGLVCQTNITCFLRNDNEGQDYEVNQTLRERQLYTKNDSLTGTDFGMTDDGPSDAYIGHLDYGGGVECDALFPDWGRPDVAEWWGNNYKKLFSIGLDFVWQDMTVPAMMPHKIGDDINVKPDGNWPNADDPSNGQYNWKTYHPQVLVTDMRYENHGREPMVTQRNIHAYTLCESTRKEGIVENADTLTKFRRSYIISRGGYIGNQHFGGMWVGDNSTTSNYIQMMIANNINMNMSCLPLVGSDIGGFTSYDNENQRTPCTGDLMVRYVQAGCLLPWFRNHYDRWIESKDHGKDYQELYMYPNEMDTLRKFVEFRYRWQEVLYTAMYQNAAFGKPIIKAASMYNNDSNVRRAQNDHFLLGGHDGYRILCAPVVWENSTERELYLPVLTQWYKFGPDFDTKPLEGAMNGGDRIYNYPVPQSESPIFVREGAILPTRYTLNGENKSLNTYTDEDPLVFEVFPLGNNRADGMCYLDDGGVTTNAEDNGKFSVVKVAAEQDGGTETITFTNDCYEYVFGGPFYVRVRGAQSPSNIHVSSGAGSQDMKVSSATSRAALFNDGENGDFWVDQETDSLWLKLPNVVLPDAVITIT

SEQ ID NO: 2 - protein, 1091 aa, Gracilariopsis lemaneiformis
MYPTLTFVAPSALGARTFTCVGIFRSHILIHSVVPAVRLAVRKSNRLNVSMSALFDKPTAVTGGKDNPDNINYTTYDYVPVWRFDPLSNTNWFAAGSSTPGDIDDWTATMNVNFDRIDNPSFTLEKPVQVQVTSYKNNCFRVRFNPDGPIRDVDRGPILQQQLNWIRKQEQSKGFDPKMGFTKEGFLKFETKDLNVIIYGNFKTRVTRKRDGKGIMENNEVPAGSLGNKCRGLMFVDRLYGTAIASVNENYRNDPDRKEGFYGAGEVNCEFWDSEQNRNKYILERTGIAMTNYNYDNYNYNQSDLIAPGYPSDPNFYIPMYFAAPWVVVKGCSGNSDEQYSYGWFMDNVSQTYMNTGGTSWNCGEENLAYMGAQCGPFDQHFVYGDGDGLEDVVQAFSLLQGKEFENQVLNKRAVMPPKYVFGYFQGVFGIASLLREQRPEGGNNISVQEIVEGYQSNNFPLEGLAVDVDMQQDLRVFTTKIEFWTANKVGTGGDSNNKSVFEWAHDKGLVCQTNVTCFLRNDNGGADYEVNQTLREKGLYTKNDSLTNTNFGTTNDGPSDAYIGHLDYGGGGNCDALFPDWGRPGVAEWWGDNYSKLFKIGLDFVWQDMTVPAMMPHKVGDAVDTRSPYGWPNENDPSNGRYNWKSYHPQVLVTDMRYENHGREPMFTQRNMHAYTLCESTRKEGIVANADTLTKFRRSYIISRGGYIGNQHFGGMWVGDNSSSQRYLQMMIANIVNMNMSCLPLVGSDIGGFTSYDGRNVCPGDLMVRFVQAGCLLPWFRNHYGRLVEGKQEGKYYQELYMYKDEMATLRKFIEFRYRWQEVLYTAMYQNAAFGKPIIKAASMYDNDRNVRGAQDDHFLLGGHDGYRILCAPVVWENTTSRDLYLPVLTKWYKFGPDYDTKRLDSALDGGQMIKNYSVPQSDSPIFVREGAILPTRYTLDGSNKSMNTYTDKDPLVFEVFPLGNNRADGMCYLDDGGITTDAEDHGKFSVINVEALRKGVTTTIKFAYDTYQYVFDGPFYVRIRNLTTASKINVSSGAGEEDMTPTSANSRAALFSDGGVGEYWADNDTSSLWMKLPNLVLQDAVITIT

SEQ ID NO: 3 - protein, 1066 aa, Morchella costata
MAGFSDPLNFCKAEDYYSVALDWKGPQKIIGVDTTPPKSTKFPKNWHGVNLRFDDGTLGVVQFIRPCVWRVRYDPGFKTSDEYGDENTRTIVQDYMSTLSNKLDTYRGLTWETKCEDSGDFFTFSSKVTAVEKSERTRNKVGDGLRIHLWKSPFRIQVVRTLTPLKDPYPIPNVAAAEARVSDKVVWQTSPKTFRKNLHPQHKMLKDTVLDIVKPGHGEYVGWGEMGGIQFMKEPTFMNYFNFDNMQYQQVYAQGALDSREPLYHSDPFYLDVNSNPEHKNITATFIDNYSQIAIDFGKTNSGYIKLGTRYGGIDCYGISADTVPEIVRLYTGLVGRSKLKPRYILGAHQACYGYQQESDLYSVVQQYRDCKFPLDGIHVDVDVQDGFRTFTTNPHTFPNPKEMFTNLRNNGIKCSTNITPVISINNREGGYSTLLEGVDKKYFIMDDRYTEGTSGNAKDVRYMYYGGGNKVEVDPNDVNGRPDFKDNYDFPANFNSKQYPYHGGVSYGYGNGSAGFYPDLNRKEVRIWWGMQYKYLFDMGLEFVWQDMTTPAIHTSYGDMKGLPTRLLVTSDSVTNASEKKLAIETWALYSYNLHKATWHGLSRLESRKNKRNFILGRGSYAGAYRFAGLWTGDNASNWEFWKISVSQVLSLGLNGVCIAGSDTGGFEPYRDANGVEEKYCSPELLIRWYTGSFLLPWLRNHYVKKDRKWFQEPYSYPKHLETHPELADQAWLYKSVLEICRYYVELRYSLIQLLYDCMFQNVVDGMPITRSMLLTDTEDTTFFNESQKFLDNQYMAGDDILVAPILHSRKEIPGENRDVYLPLYHTWYPSNLRPWDDQGVALGNPVEGGSVINYTARIVAPEDYNLFHSVVPVYVREGAIIPQIEVRQWTGQGGANRIKFNIYPGKDKEYCTYLDDGVSRDSAPEDLPQYKETHEQSKVEGAEIAKQIGKKTGYNISGTDPEAKGYHRKVAVTQTSKDKTRTVTIEPKHNGYDPSKEVGDYYTIILWYAPGFDGSIVDVSKTTVNVEGGVEHQVYKNSDLHTVVIDVKEVIGTTKSVKITCTAA

SEQ ID NO: 4 - protein, 1070 aa, Morchella vulgaris
MAGLSDPLNFCKAEDYYAAAKGWSGPQKIIRYDQTPPQGTKDPKSWHAVNLPFDDGTMCVVQFVRPCVWRVRYDPSVKTSDEYGDENTRTIVQDYMTTLVGNLDIFRGLTWVSTLEDSGEYYTFKSEVTAVDETERTRNKVGDGLKIYLWKNPFRIQVVRLLTPLVDPFPIPNVANATARVADKVVWQTSPKTFRKNLHPQHKMLKDTVLDIIKPGHGEYVGWGEMGGIEFMKEPTFMNYFNFDNMQYQQVYAQGALDSREPLYHSDPFYLDVNSNPEHKNITATFIDNYSQIAIDFGKTNSGYIKLGTRYGGIDCYGISADTVPEIVRLYTGLVGRSKLKPRYILGAHQACYGYQQESDLHAVVQQYRDTKFPLDGLHVDVDFQDNFRTFTTNPITFPNPKEMFTNLRNNGIKCSTNITPVISIRDRPNGYSTLNEGYDKKYFIMDDRYTEGTSGDPQNVRYSFYGGGNPVEVNPNDVWARPDFGDNYDFPTNFNCKDYPYHGGVSYGYGNGTPGYYPDLNREEVRIWWGLQYEYLFNMGLEFVWQDMTTPAIHSSYGDMKGLPTRLLVTADSVTNASEKKLAIESWALYSYNLHKATFHGLGRLESRKNKRNFILGRGSYAGAYRFAGLWTGDNASTWEFWKISVSQVLSLGLNGVCIAGSDTGGFEPARTEIGEEKYCSPELLIRWYTGSFLLPWLRNHYVKKDRKWFQEPYAYPKHLETHPELADQAWLYKSVLEICRYWVELRYSLIQLLYDCMFQNVVDGMPLARSMLLTDTEDTTFFNESQKFLDNQYMAGDDILVAPILHSRNEVPGENRDVYLPLFHTWYPSNLRPWDDQGVALGNPVEGGSVINYTARIVAPEDYNLFHNVVPVYIREGAIIPQIQVRQWIGEGGPNPIKFNIYPGKDKEYVTYLDDGVSRDSAPDDLPQYREAYEQAKVEGKDVQKQLAVIQGNKTNDFSASGIDKEAKGYHRKVSIKQESKDKTRTVTIEPKHNGYDPSKEVGNYYTIILWYAPGFDGSIVDVSQATVNIEGGVECEIFKNTGLHTVVVNVKEVIGTTKSVKITCTTA

SEQ ID NO: 5 - protein, 1092 aa, Gracilariopsis lemaneiformis
MFPTLTFIAPSALAASTFVGADIRSGIRIQSALPAVRNAVRRSKHYNVSMTALSDKQTAISIGPDNPDGINYQNYDYIPVAGFTPLSNTNWYAAGSSTPGGITDWTATMNVKFDRIDNPSYSNNHPVQIQVTSYNNNSFRIRFNPDGPIRDVSRGPILKQQLTWIRNQELAQGCNPNMSFSPEGFLSFETKDLNVIIYGNCKMRVTKKDGYLVMENDECNSQSDGNKCRGLMYVDRLYGNAIASVQTNFHKDTSRNEKFYGAGEVNCRYEEQGKAPTYVLERSGLAMTNYNYDNLNYNQPDVVPPGYPDHPNYYIPMYYAAPWLVVQGCAGTSKQYSYGWFMDNVSQSYMNTGDTAWNCGQENLAYMGAQYGPFDQHFVYGDGDGLEDVVKAFSFLQGKEFEDKKLNKRSVMPPKYVFGFFQGVFGALSLLKQNLPAGENNISVQEIVEGYQDNDYPFEGLAVDVDMQDDLRVFTTKPEYWSANMVGEGGDPNNRSVFEWAHDRGLVCQTNVTCFLRNDNSGKPYEVNQTLREKQLYTKNDSLNNTDFGTTSDGPGDAYIGHLDYGGGVECDAIFPDWGRPDVAQWWGENYKKLFSIGLDFVWQDMTVPAMMPHRLGDAVNKNSGSSAPGWPNENDPSNGRYNWKSYHPQVLVTDMRYGAEYGREPMVSQRNIHAYTLCESTRREGIVGNADSLTKFRRSYIISRGGYIGNQHFGGMWVGDNSATESYLQMMLANIINMNMSCLPLVGSDIGGFTQYNDAGDPTPEDLMVRFVQAGCLLPWFRNHYDRWIESKKHGKKYQELYMYPGQKDTLKKFVEFRYRWQEVLYTAMYQNATTGEPIIKAAPMYNNDVNVYKSQNDHFLLGGHDGYRILCAPVVRENATSREVYLPVYSKWFKFGPDFDTKPLENEIQGGQTLYNYAAPLNDSPIFVREGTILPTRYTLDGVNKSINTYTDNDPLVFELFPLENNQAHGLFYHDDGGVTTNAEDFGKYSVISVKAAQEGSQMSVKFDNEVYEHQWGASFYVRVRNMGAPSNINVSSQIGQQDMQQSSVSSRAQMFTSANDGEYWVDQSTNSLWLKLPGAVIQDAAITVR

SEQ ID NO: 6 - protein, 571 aa, Gracilariopsis lemaneiformis
MTNYNYDNLNYNQPDLIPPGHDSDPDYYIPMYFAAPWVIAHGYRGTSDQYSYGWFLDNVSQSYTNTGDDAWAGQKDLAYMGAQCGPFDQHFVYEAGDGLEDVVTAFSYLQGKEYENQGLNIRSAMPPKYVFGFFQGVFGATSLLRDNLPAGENNVSLEEIVEGYQNQNVPFEGLAVDVDMQDDLRVFTTRPAFWTANKVGEGGDPNNKSVFEWAHDRGLVCQTNVTCFLKNEKNPYEVNQSLREKQLYTKSDSLDNIDFGTTPDGPSDAYIGHLDYGGGVECDALFPDWGRPDVAQWWGDNYKKLFSIGLDFVWQDMTVPAMMPHRLGDPVGTNSGETAPGWPNDKDPSNGRYNWKSYHPQVLVTDMRYDDYGRDPIVTQRNLHAYTLCESTRREGIVGNADSLTKFRRSYIISRGGYIGNQHFGGMWVGDNSSTEDYLAMMVINVINMNMSGVPLVGSDIGGFTEHDKRNPCTPDLMMRFVQAGCLLPWFRNHYDRWIESKKHGKNYQELYMYRDHLDALRSFVELRYRWQEVLYTAMYQNALNGKPIIKTVSMYNNDMNVKDAQNDHFL

SEQ ID NO: 7 - DNA, 3267 bp, Gracilariopsis lemaneiformis
atgttttcaacccttgcgtttgtcgcacctagtgcgctgggagccagtaccttcgtaggggcggaggtcaggtcaaatgttcgtatccattccgcttttccagctgtgcacacagctactcgcaaaaccaatcgcctcaatgtatccatgaccgcattgtccgacaaacaaacggctactgcgggtagtacagacaatccggacggtatcgactacaagacctacgattacgtcggagtatggggtttcagccccctctccaacacgaactggtttgctgccggctcttctaccccgggtggcatcactgattggacggctacaatgaatgtcaacttcgaccgtatcgacaatccgtccatcactgtccagcatcccgttcaggttcaggtcacgtcatacaacaacaacagctacagggttcgcttcaaccctgatggccctattcgtgatgtgactcgtgggcctatcctcaagcagcaactagattggattcgaacgcaggagctgtcagagggatgtgatcccggaatgactttcacatcagaaggtttcttgacttttgagaccaaggatctaagcgtcatcatctacggaaatttcaagaccagagttacgagaaagtctgacggcaaggtcatcatggaaaatgatgaagttggaactgcatcgtccgggaacaagtgccggggattgatgttcgttgatagattatacggtaacgctatcgcttccgtcaacaagaacttccgcaacgacgcggtcaagcaggagggattctatggtgcaggtgaagtcaactgtaagtaccaggacacctacatcttagaacgcactggaatcgccatgacaaattacaactacgataacttgaactataaccagtgggaccttagacctccgcatcatgatggtgccctcaacccagactattatattccaatgtactacgcagcaccttggttgatcgttaatggatgcgccggtacttcggagcagtactcgtatggatggttcatggacaatgtctctcaatcttacatgaatactggagatactacctggaattctggacaagaggacctggcatacatgggcgcgcagtatggaccatttgaccaacattttgtttacggtgctgggggtgggatggaatgtgtggtcacagcgttctctcttctacaaggcaaggagttcgagaaccaagttctcaacaaacgttcagtaatgcctccgaaatacgtctttggtttcttccagggtgttttcgggacttcttccttgttgagagcgcatatgccagcaggtgagaacaacatctcagtcgaagaaattgtagaaggttatcaaaacaacaatttccctttcgaggggctcgctgtggacgtggatatgcaagacaacttgcgggtgttcaccacgaagggcgaattttggaccgcaaacagggtgggtactggcggggatccaaacaaccgatcggtttttgaatgggcacatgacaaaggccttgtttgtcagacaaatataacttgcttcctgaggaatgataacgaggggcaagactacgaggtcaatcagacgttaagggagaggcagttgtacacgaagaacgactccctgacgggtacggattttggaatgaccgacgacggccccagcgatgcgtacatcggtcatctggactatgggggtggagtagaatgtgatgcacttttcccagactggggacggcctgacgtggccgaatggtggggaaataactataagaaactgttcagcattggtctcgacttcgtctggcaagacatgactgttccagcaatgatgccgcacaaaattggcgatgacatcaatgtgaaaccggatgggaattggccgaatgcggacgatccgtccaatggacaatacaactggaagacgtaccatccccaagtgcttgtaactgatatgcgttatgagaatcatggtcgggaaccgatggtcactcaacgcaacattcatgcgtatacactgtgcgagtctactaggaaggaagggatcgtggaaaacgcagacactctaacgaagttccgccgtagctacattatcagtcgtggtggttacattggtaaccagcatttcgggggtatgtgggtgggagacaactctactacatcaaactacatccaaatgatgattgccaacaatattaacatgaatatgtcttgcttgcctctcgtcggctccgacattggaggattcacctcatacgacaatgagaatcagcgaacgccgtgtaccggggacttgatggtgaggtatgtgcaggcgggctgcctgttgccgtggttcaggaaccactatgataggtggatcgagtccaaggaccacggaaaggactaccaggagctgtacatgtatccgaatgaaatggatacgttgaggaagttcgttgaattccgttatcgctggcaggaagtgttgtacacggccatgtaccagaatgcggctttcggaaagccgattatcaaggctgcttcgatgtacaataacgactcaaacgttcgcagggcgcagaacgatcatttccttcttggtggacatgatggatatcgcattctgtgcgcgcctgttgtgtgggagaattcgaccgaacgcgaattgtacttgcccgtgctgacccaatggtacaaattcggtcccgactttgacaccaagcctctggaaggagcgatgaacggaggggaccgaatttacaactaccctgtaccgcaaagtgaatcaccaatcttcgtgagagaaggtgcgattctccctacccgctacacgttgaacggtgaaaacaaatcattgaacacgtacacggacgaagatccgttggtgtttgaagtattccccctcggaaacaaccgtgccgacggtatgtgttatcttgatgatggcggtgtgaccaccaatgctgaagacaatggcaagttctctgtcgtcaaggtggcagcggagcaggatggtggtacggagacgataacgtttacgaatgattgctatgagtacgttttcggtggaccgttctacgttcgagtgcgcggcgctcagtcgccgtcgaacatccacgtgtcttctggagcgggttctcaggacatgaaggtgagctctgccacttccagggctgcgctgttcaatgacggggagaacggtgatttctgggttgaccaggagacagattctctgtggctgaagttgcccaacgttgttctcccggacgctgtgatcacaattacctaa

SEQ ID NO: 8 - DNA, 3276 bp, Gracilariopsis lemaneiformis
atgtatccaaccctcaccttcgtggcgcctagtgcgctaggggccagaactttcacgtgtgtgggcatttttaggtcacacattcttattcattcggttgttccagcggtgcgtctagctgtgcgcaaaagcaaccgcctcaatgtatccatgtccgctttgttcgacaaaccgactgctgttactggagggaaggacaacccggacaatatcaattacaccacttatgactacgtccctgtgtggcgcttcgaccccctcagcaatacgaactggtttgctgccggatcttccactcccggcgatattgacgactggacggcgacaatgaatgtgaacttcgaccgtatcgacaatccatccttcactctcgagaaaccggttcaggttcaggtcacgtcatacaagaacaattgtttcagggttcgcttcaaccctgatggtcctattcgcgatgtggatcgtgggcctatcctccagcagcaactaaattggatccggaagcaggagcagtcgaaggggtttgatcctaagatgggcttcacaaaagaaggtttcttgaaatttgagaccaaggatctgaacgttatcatatatggcaattttaagactagagttacgaggaagagggatggaaaagggatcatggagaataatgaagtgccggcaggatcgttagggaacaagtgccggggattgatgtttgtcgacaggttgtacggcactgccatcgcttccgttaatgaaaattaccgcaacgatcccgacaggaaagaggggttctatggtgcaggagaagtaaactgcgagttttgggactccgaacaaaacaggaacaagtacatcttagaacgaactggaatcgccatgacaaattacaattatgacaactataactacaaccagtcagatcttattgctccaggatatccttccgacccgaacttctacattcccatgtattttgcagcaccttgggtagttgttaagggatgcagtggcaacagcgatgaacagtactcgtacggatggtttatggataatgtctcccaaacttacatgaatactggtggtacttcctggaactgtggagaggagaacttggcatacatgggagcacagtgcggtccatttgaccaacattttgtgtatggtgatggagatggtcttgaggatgttgtccaagcgttctctcttctgcaaggcaaagagtttgagaaccaagttctgaacaaacgtgccgtaatgcctccgaaatatgtgtttggttactttcagggagtctttgggattgcttccttgttgagagagcaaagaccagagggtggtaataacatctctgttcaagagattgtcgaaggttaccaaagcaataacttccctttagaggggttagccgtagatgtggatatgcaacaagatttgcgcgtgttcaccacgaagattgaattttggacggcaaataaggtaggcaccgggggagactcgaataacaagtcggtgtttgaatgggcacatgacaaaggccttgtatgtcagacgaatgttacttgcttcttgagaaacgacaacggcggggcagattacgaagtcaatcagacattgagggagaagggtttgtacacgaagaatgactcactgacgaacactaacttcggaactaccaacgacgggccgagcgatgcgtacattggacatctggactatggtggcggagggaattgtgatgcacttttcccagactggggtcgaccgggtgtggctgaatggtggggtgataactacagcaagctcttcaaaattggtctggatttcgtctggcaagacatgacagttccagctatgatgccacacaaagttggcgacgcagtcgatacgagatcaccttacggctggccgaatgagaatgatccttcgaacggacgatacaattggaaatcttaccatccacaagttctcgtaactgatatgcgatatgagaatcatggaagggaaccgatgttcactcaacgcaatatgcatgcgtacacactctgtgaatctacgaggaaggaagggattgttgcaaatgcagacactctaacgaagttccgccgcagttatattatcagtcgtggaggttacattggcaaccagcattttggaggaatgtgggttggagacaactcttcctcccaaagatacctccaaatgatgatcgcgaacatcgtcaacatgaacatgtcttgccttccactagttgggtccgacattggaggttttacttcgtatgatggacgaaacgtgtgtcccggggatctaatggtaagattcgtgcaggcgggttgcttactaccgtggttcagaaaccactatggtaggttggtcgagggcaagcaagagggaaaatactatcaagaactgtacatgtacaaggacgagatggctacattgagaaaattcattgaattccgttaccgctggcaggaggtgttgtacactgctatgtaccagaatgcggctttcgggaaaccgattatcaaggcagcttccatgtacgacaacgacagaaacgttcgcggcgcacaggatgaccacttccttctcggcggacacgatggatatcgtattttgtgtgcacctgttgtgtgggagaatacaaccagtcgcgatctgtacttgcctgtgctgaccaaatggtacaaattcggccctgactatgacaccaagcgcctggattctgcgttggatggagggcagatgattaagaactattctgtgccacaaagcgactctccgatatttgtgagggaaggagctattctccctacccgctacacgttggacggttcgaacaagtcaatgaacacgtacacagacaaagacccgttggtgtttgaggtattccctcttggaaacaaccgtgccgacggtatgtgttatcttgatgatggcggtattactacagatgctgaggaccatggcaaattctctgttatcaatgtcgaagccttacggaaaggtgttacgacgacgatcaagtttgcgtatgacacttatcaatacgtatttgatggtccattctacgttcgaatccgtaatcttacgactgcatcaaaaattaacgtgtcttctggagcgggtgaagaggacatgacaccgacctctgcgaactcgagggcagctttgttcagtgatggaggtgttggagaatactgggctgacaatgatacgtcttctctgtggatgaagttgccaaacctggttctgcaagacgctgtgattaccattacgtag

SEQ ID NO: 9 - DNA, 3201 bp, Morchella costata
atggcaggattttctgatcctctcaacttttgcaaagcagaagactactacagtgttgcgctagactggaagggccctcaaaaaatcattggagtagacactactcctccaaagagcaccaagttccccaaaaactggcatggagtgaacttgagattcgatgatgggactttaggtgtggttcagttcattaggccgtgcgtttggagggttagatacgaccctggtttcaagacctctgacgagtatggtgatgagaatacgaggacaattgtgcaagattatatgagtactctgagtaataaattggatacttatagaggtcttacgtgggaaaccaagtgtgaggattcgggagatttctttaccttctcatccaaggtcaccgccgttgaaaaatccgagcggacccgcaacaaggtcggcgatggcctcagaattcacctatggaaaagccctttccgcatccaagtagtgcgcaccttgacccctttgaaggatccttaccccattccaaatgtagccgcagccgaagcccgtgtgtccgacaaggtcgtttggcaaacgtctcccaagacattcagaaagaacctgcatccgcaacacaagatgctaaaggatacagttcttgacattgtcaaacctggacatggcgagtatgtggggtggggagagatgggaggtatccagtttatgaaggagccaacattcatgaactattttaacttcgacaatatgcaataccagcaagtctatgcccaaggtgctctcgattctcgcgagccactgtaccactcggatcccttctatcttgatgtgaactccaacccggagcacaagaatatcacggcaacctttatcgataactactctcaaattgccatcgactttggaaagaccaactcaggctacatcaagctgggaaccaggtatggtggtatcgattgttacggtatcagtgcggatacggtcccggaaattgtacgactttatacaggtcttgttggacgttcaaagttgaagcccagatatattctcggggcccatcaagcctgttatggataccaacaggaaagtgacttgtattctgtggtccagcagtaccgtgactgtaaatttccacttgacgggattcacgtcgatgtcgatgttcaggacggcttcagaactttcaccaccaacccacacactttccctaaccccaaagagatgtttactaacttgaggaataatggaatcaagtgctccaccaatatcactcctgttatcagcattaacaacagagagggtggatacagtaccctccttgagggagttgacaaaaaatactttatcatggacgacagatataccgagggaacaagtgggaatgcgaaggatgttcggtacatgtactacggtggtggtaataaggttgaggtcgatcctaatgatgttaatggtcggccagactttaaagacaactatgacttccccgcgaacttcaacagcaaacaatacccctatcatggtggtgtgagctacggttatgggaacggtagtgcaggtttttacccggacctcaacagaaaggaggttcgtatctggtggggaatgcagtacaagtatctcttcgatatgggactggaatttgtgtggcaagacatgactaccccagcaatccacacatcatatggagacatgaaagggttgcccacccgtctactcgtcacctcagactccgtcaccaatgcctctgagaaaaagctcgcaattgaaacttgggctctctactcctacaatctccacaaagcaacttggcatggtcttagtcgtctcgaatctcgtaagaacaaacgaaacttcatcctcgggcgtggaagttatgccggagcctatcgttttgctggtctctggactggggataatgcaagtaactgggaattctggaagatatcggtctctcaagttctttctctgggcctcaatggtgtgtgcatcgcggggtctgatacgggtggttttgaaccctaccgtgatgcaaatggggtcgaggagaaatactgtagcccagagctactcatcaggtggtatactggttcattcctcttgccgtggctcaggaaccattatgtcaaaaaggacaggaaatggttccaggaaccatactcgtaccccaagcatcttgaaacccatccagaactcgcagaccaagcatggctctataaatccgttttggagatctgtaggtactatgtggagcttagatactccctcatccaactactttacgactgcatgtttcaaaacgtagtcgacggtatgccaatcaccagatctatgctcttgaccgatactgaggataccaccttcttcaacgagagccaaaagttcctcgacaaccaatatatggctggtgacgacattcttgttgcacccatcctccacagtcgcaaagaaattccaggcgaaaacagagatgtctatctccctctttaccacacctggtacccctcaaatttgagaccatgggacgatcaaggagtcgctttggggaatcctgtcgaaggtggtagtgtcatcaattatactgctaggattgttgcacccgaggattataatctcttccacagcgtggtaccagtctacgttagagagggtgccatcatcccgcaaatcgaagtacgccaatggactggccaggggggagccaaccgcatcaagttcaacatctaccctggaaaggataaggagtactgtacctatcttgatgatggtgttagccgtgatagtgcgccggaagacctcccacagtacaaagagacccacgaacagtcgaaggttgaaggcgcggaaatcgcaaagcagattggaaagaagacgggttacaacatctcaggaaccgacccagaagcaaagggttatcaccgcaaagttgctgtcacacaaacgtcaaaagacaagacgcgtactgtcactattgagccaaaacacaatggatacgacccttccaaagaggtgggtgattattataccatcattctttggtacgcaccaggtttcgatggcagcatcgtcgatgtgagcaagacgactgtgaatgttgagggtggggtggagcaccaagtttataagaactccgatttacatacggttgttatcgacgtgaaggaggtgatcggtaccacaaagagcgtcaagatcacatgtactgccgcttaa

SEQ ID NO: 10 - DNA, 3213 bp, Morchella vulgaris
atggcaggattatccgaccctctcaatttctgcaaagcagaggactactacgctgctgccaaaggctggagtggccctcagaagatcattcgctatgaccagacccctcctcagggtacaaaagatccgaaaagctggcatgcggtaaaccttcctttcgatgacgggactatgtgtgtagtgcaattcgtcagaccctgtgtttggagggttagatatgaccccagtgtcaagacttctgatgagtacggcgatgagaatacgaggactattgtacaagactacatgactactctggttggaaacttggacattttcagaggtcttacgtgggtttctacgttggaggattcgggcgagtactacaccttcaagtccgaagtcactgccgtggacgaaaccgaacggactcgaaacaaggtcggcgacggcctcaagatttacctatggaaaaatccctttcgcatccaggtagtgcgtctcttgacccccctggtggaccctttccccattcccaacgtagccaatgccacagcccgtgtggccgacaaggttgtttggcagacgtccccgaagacgttcaggaaaaacttgcatccgcagcataagatgttgaaggatacagttcttgatattatcaagccggggcacggagagtatgtgggttggggagagatgggaggcatcgagtttatgaaggagccaacattcatgaattatttcaactttgacaatatgcaatatcagcaggtctatgcacaaggcgctcttgatagtcgtgagccgttgtatcactctgatcccttctatctcgacgtgaactccaacccagagcacaagaacattacggcaacctttatcgataactactctcagattgccatcgactttgggaagaccaactcaggctacatcaagctgggtaccaggtatggcggtatcgattgttacggtatcagcgcggatacggtcccggagattgtgcgactttatactggacttgttgggcgttcgaagttgaagcccaggtatattctcggagcccaccaagcttgttatggataccagcaggaaagtgacttgcatgctgttgttcagcagtaccgtgacaccaagtttccgcttgatgggttgcatgtcgatgtcgactttcaggacaatttcagaacgtttaccactaacccgattacgttccctaatcccaaagaaatgtttaccaatctaaggaacaatggaatcaagtgttccaccaacatcacccctgttatcagtatcagagatcgcccgaatgggtacagtaccctcaatgagggatatgataaaaagtacttcatcatggatgacagatataccgaggggacaagtggggacccgcaaaatgttcgatactctttttacggcggtgggaacccggttgaggttaaccctaatgatgtttgggctcggccagactttggagacaattatgacttccctacgaacttcaactgcaaagactacccctatcatggtggtgtgagttacggatatgggaatggcactccaggttactaccctgaccttaacagagaggaggttcgtatctggtggggattgcagtacgagtatctcttcaatatgggactagagtttgtatggcaagatatgacaaccccagcgatccattcatcatatggagacatgaaagggttgcccacccgtctgctcgtcaccgccgactcagttaccaatgcctctgagaaaaagctcgcaattgaaagttgggctctttactcctacaacctccataaagcaaccttccacggtcttggtcgtcttgagtctcgtaagaacaaacgtaacttcatcctcggacgtggtagttacgccggtgcctatcgttttgctggtctctggactggagataacgcaagtacgtgggaattctggaagatttcggtctcccaagttctttctctaggtctcaatggtgtgtgtatagcggggtctgatacgggtggttttgagcccgcacgtactgagattggggaggagaaatattgcagtccggagctactcatcaggtggtatactggatcattccttttgccatggcttagaaaccactacgtcaagaaggacaggaaatggttccaggaaccatacgcgtaccccaagcatcttgaaacccatccagagctcgcagatcaagcatggctttacaaatctgttctagaaatttgcagatactgggtagagctaagatattccctcatccagctcctttacgactgcatgttccaaaacgtggtcgatggtatgccacttgccagatctatgctcttgaccgatactgaggatacgaccttcttcaatgagagccaaaagttcctcgataaccaatatatggctggtgacgacatccttgtagcacccatcctccacagccgtaacgaggttccgggagagaacagagatgtctatctccctctattccacacctggtacccctcaaacttgagaccgtgggacgatcagggagtcgctttagggaatcctgtcgaaggtggcagcgttatcaactacactgccaggattgttgccccagaggattataatctcttccacaacgtggtgccggtctacatcagagagggtgccatcattccgcaaattcaggtacgccagtggattggcgaaggagggcctaatcccatcaagttcaatatctaccctggaaaggacaaggagtatgtgacgtaccttgatgatggtgttagccgcgatagtgcaccagatgacctcccgcagtaccgcgaggcctatgagcaagcgaaggtcgaaggcaaagacgtccagaagcaacttgcggtcattcaagggaataagactaatgacttctccgcctccgggattgataaggaggcaaagggttatcaccgcaaagtttctatcaaacaggagtcaaaagacaagacccgtactgtcaccattgagccaaaacacaacggatacgacccctctaaggaagttggtaattattataccatcattctttggtacgcaccgggctttgacggcagcatcgtcgatgtgagccaggcgaccgtgaacatcgagggcggggtggaatgcgaaattttcaagaacaccggcttgcatacggttgtagtcaacgtgaaagaggtgatcggtaccacaaagtccgtcaagatcacttgcactaccgcttag

SEQ ID NO: 11 - DNA, 3279 bp, Gracilariopsis lemaneiformis
atgtttcctaccctgaccttcatagcgcccagcgcgctggccgccagcacctttgtgggcgcggatatccgatcgggcattcgcattcaatccgctcttccggccgtgcgcaacgctgtgcgcaggagcaaacattacaatgtatccatgaccgcattgtctgacaagcaaaccgctatcagtattggccctgacaatccggacggtatcaactaccaaaactacgattacatccctgtagcgggctttacgcccctctccaacaccaactggtatgctgccggctcttccactccgggcggcatcaccgactggaccgctaccatgaatgtcaaattcgaccgcattgacaatccgtcgtactccaataaccatcctgttcagattcaggtcacgtcgtacaacaacaacagcttcaggattcgcttcaaccctgatggccccattcgtgacgtctctcgaggacctatcctgaaacagcaactcacttggattcgaaaccaggagctggcgcagggatgtaatccgaacatgagcttctctcctgaaggttttttgtcttttgaaaccaaagacctaaacgttataatctacggcaactgcaagatgagagtcacgaagaaggatggctacctcgtcatggagaatgacgagtgcaactcgcaatcagatggcaataagtgtagaggattgatgtacgttgaccggctatacggtaatgctattgcttccgtacaaacgaattttcacaaagacacttctcggaacgagaaattctatggtgcaggtgaagtcaactgtcgctatgaggagcagggtaaggcgccgacttatgttctagaacgctctggactcgccatgaccaattacaattacgacaacttgaactacaaccaaccagacgtcgttcctccaggttatcccgaccatcccaactactacattccaatgtactacgcagcaccgtggttggtcgttcagggatgcgcggggacatcgaagcaatactcgtacggttggtttatggacaatgtctctcagtcgtacatgaacactggagatacggcgtggaactgcggacaggaaaacctggcatacatgggcgcgcaatacgggccatttgatcagcactttgtgtatggtgatggagatggccttgaagatgtcgtcaaagcgttctcctttcttcaaggaaaggagttcgaagacaaaaaactcaacaagcgttctgtaatgcctccgaagtacgtgtttggtttcttccagggtgttttcggtgcactttcactgttgaagcagaatctgcctgccggagagaacaacatctcagtgcaagagattgtggagggttaccaggataacgactacccctttgaagggctcgcggtagatgttgatatgcaagatgatctgcgagtgtttactaccaaaccagaatattggtcggcaaacatggtaggcgaaggcggtgatcctaataacagatcagtctttgaatgggcacatgacaggggccttgtctgtcagacgaacgtaacttgcttcttgaggaacgataacagtgggaaaccatacgaagtgaatcagacattgagggagaaacagttgtatacgaagaatgattccttgaacaacaccgattttggaactacctcggatgggcctggcgatgcgtacattggacatttggactatggtggtggagtggagtgtgatgcaatcttcccagactggggtcgaccagacgtggctcaatggtggggagaaaactacaagaagctgttcagcattggtctcgatttcgtgtggcaggatatgacggtacctgcgatgatgccgcaccgactcggtgatgctgtcaacaaaaattccggtagttcggcgccgggctggccgaatgagaacgatccatccaacggacgatacaactggaaatcttatcatccgcaagtgctcgtgaccgacatgcgctatggtgcagagtatggaagggaaccgatggtgtctcaacgcaacattcacgcctacactctttgtgaatctaccagacgggagggaattgtgggaaacgcagacagtttgaccaagttccgccgcagttacatcatcagtcgaggaggttacatcggtaaccagcatttcggagggatgtgggttggggacaacagtgccacagaatcctacctccaaatgatgttggcgaacattatcaacatgaatatgtcgtgcctcccgctagttggctctgatattggcgggttcacccagtacaatgatgcgggcgacccaacccccgaggatttgatggtaagattcgtgcaggctggctgtctgctaccgtggttcagaaaccactatgacaggtggattgagtccaagaagcacgggaagaaataccaggagttatacatgtacccggggcaaaaggatacgttgaagaagttcgttgaattccgctaccgctggcaggaggttttgtacacagccatgtaccaaaatgctaccactggagagccgatcatcaaggcggcgcccatgtacaacaacgacgtcaacgtgtataaatcgcagaatgatcatttccttctcggtggacatgacggctatcgtattctctgcgcacctgttgtgcgcgaaaatgcgacaagtcgcgaagtgtacctgcctgtgtatagcaagtggttcaaattcggaccggactttgacactaagcccttggaaaatgagattcaaggaggtcagacgctttataattacgctgcaccgctgaacgattcgccgatatttgtgagggaagggactattcttccgacacggtacacgctggacggtgtgaacaaatctatcaacacgtacacagacaatgatccgcttgtatttgagctgttccctctcgaaaacaaccaggcgcatggcttgttctatcatgatgatggcggtgtcaccaccaacgctgaagactttggcaagtattctgtgatcagtgtgaaggccgcgcaggaaggttctcaaatgagtgtcaagtttgacaatgaagtttatgaacaccaatggggagcatcgttctatgttcgtgttcgtaatatgggtgctccgtctaacatcaacgtatcttctcagattggtcaacaggacatgcaacagagctccgtgagttccagggcgcaaatgttcactagtgctaacgatggcgagtactgggttgaccagagcacgaactcgttgtggctcaagttgcctggtgcagttatccaagacgctgcgatcactgttcgttga

SEQ ID NO: 12 - DNA, 1712 bp, Gracilariopsis lemaneiformis
atgacaaactataattatgacaatttgaactacaatcaaccggacctcatcccacctggccatgattcagatcctgactactatattccgatgtactttgcggcaccatgggtgatcgcacatggatatcgtggcaccagcgaccagtactcttatggatggtttttggacaatgtatcccagtcctacacaaacactggcgatgatgcatgggctggtcagaaggatttggcgtacatgggggcacaatgtgggcctttcgatcaacattttgtgtatgaggctggagatggacttgaagacgttgtgaccgcattctcttatttgcaaggcaaggaatatgagaaccagggactgaatatacgttctgcaatgcctccgaagtacgttttcggatttttccaaggcgtattcggagccacatcgctgctaagggacaacttacctgccggcgagaacaacgtctctttggaagaaattgttgaaggatatcaaaatcagaacgtgccatttgaaggtcttgctgtggatgttgatatgcaagatgacttgagagtgttcactacgagaccagcgttttggacggcaaacaaggtgggggaaggcggtgatccaaacaacaagtcagtgtttgagtgggcacatgacaggggccttgtctgccagacgaatgtaacttgcttcttgaagaacgagaaaaatccttacgaagtgaatcagtcattgagggagaagcagttgtatacgaagagtgattccttggacaacattgattttggaactactccagatgggcctagcgatgcgtacattggacacttagactacggtggtggtgtggagtgtgatgcactattcccagactggggtcgaccagacgtggctcaatggtggggcgataactacaagaaactattcagcattggtctcgatttcgtctggcaagatatgacggtacctgcgatgatgccgcaccgactcggtgaccctgtcggcacaaattccggtgagacggcgccgggctggccgaatgataaggatccatccaacggacgatacaattggaagtcttaccatccgcaagtgctcgtgactgacatgaggtatgacgattacggaagagatcccattgttacgcaacgcaatctccatgcctacactctttgtgagtctactaggagggaaggcattgttggaaacgcagatagtctgacgaagttccgccgcagctatattatcagtcgtggaggctacatcggtaatcagcactttggtgggatgtgggtaggagacaactcttctacggaagactacctcgcaatgatggttatcaacgttatcaacatgaacatgtccggtgtcccgctcgttggttccgatattggaggtttcacggagcatgacaagagaaacccttgcacaccggacttgatgatgagatttgtgcaggctggatgcttgctaccgtggttcaggaaccactacgataggtggatcgagagcaagaaacacggaaagaactaccaagagttgtacatgtaccgcgaccacttggacgccttgagaagttttgtggaactccgctatcgctggcaggaagtgttatacacagccatgtatcagaatgctttgaacgggaagccgatcatcaaaacggtctccatgtacaacaacgatatgaacgtcaaagatgctcagaatgaccacttcct

SEQ ID NO: 13 - protein, 180 aa, Gracilariopsis lemaneiformis
FVWQDMTVPAMMPHKLGDAVNTRPPGDWPNAGDPSNGRYNWKSYHPQVLVTDMRYEDHGREPFVTQRNLHAYTLCESTRKEGIVGNADSLTKFRRSYIISRGGYIGNQHFGGMWVGDNSSTEDYLAMMIINVINMNMSGVPLVGSDIGGFTEHDKRNPCTPDLMMRFVQAGCLLPWFRNH

SEQ ID NO: 14 - protein, 163 aa, Anthracobia melaloma
FVWQDMTTPSIGEAYGDMKGFPSRPNPQLRRCQKEAKQAVGHRDLGSLFLQPPQTPPSTGLGRQKGRENKRNFIIGRGSFRRHVSIRGAVDWDNASTGISGGSRFRRVLSMGLSGVSISGSDDTGGFEPAKKYPGTDNDEEEKYCSPELLIRWYSGSFLLPWL

SEQ ID NO: 15 - protein, 1092 aa, Gracilariopsis lemaneiformis; identical to SEQ ID NO: 5

SEQ ID NO: 16 - DNA, 3412 bp, Gracilariopsis lemaneiformis
atgtttcctaccctgaccttcatagcgcccagcgcgctggccgccagcacctttgtgggcgcggatatccgatcgggcattcgcattcaatccgctcttccggccgtgcgcaacgctgtgcgcaggagcaaacattacaatgtatccatgaccgcattgtctgacaagcaaaccgctatcagtattggccctgacaatccggacggtatcaactaccaaaactacgattacatccctgtagcgggctttacgcccctctccaacaccaactggtatgctgccggctcttccactccgggcggcatcaccgactggaccgctaccatgaatgtcaaattcgaccgcattgacaatccgtcgtactccaataaccatcctgttcagattcaggtcacgtcgtacaacaacaacagcttcaggattcgcttcaaccctgatggccccattcgtgacgtctctcgaggacctatcctgaaacagcaactcacttggattcgaaaccaggagctggcgcagggatgtaatccgaacatgagcttctctcctgaaggttttttgtcttttgaaaccaaagacctaaacgttataatctacggcaactgcaagatgagagtcacgaagaaggatggctacctcgtcatggagaatgacgagtgcaactcgcaatcagatggcaataagtgtagaggattgatgtacgttgaccggctatacggtaatgctattgcttccgtacaaacgaattttcacaaagacacttctcggaacgagaaattctatggtgcaggtgaagtcaactgtcgctatgaggagcagggtaaggcgccgacttatgttctagaacgctctggactcgccatgaccaattacaattacgacaacttgaactacaaccaaccagacgtcgttcctccaggttatcccgaccatcccaactactacattccaatgtactacgcagcaccgtggttggtcgttcagggatgcgcggggacatcgaagcaatactcgtacggttggtttatggacaatgtctctcagtcgtacatgaacactggagatacggcgtggaactgcggacaggaaaacctggcatacatgggcgcgcaatacgggccatttgatcagcactttgtgtatggtgatggagatggccttgaagatgtcgtcaaagcgttctcctttcttcaaggaaaggagttcgaagacaaaaaactcaacaagcgttctgtaatgcctccgaagtacgtgtttggtttcttccagggtgttttcggtgcactttcactgttgaagcagaatctgcctgccggagagaacaacatctcagtgcaagagattgtggagggttaccaggataacgactacccctttgaagggctcgcggtagatgttgatatgcaagatgatctgcgagtgtttactaccaaaccagaatattggtcggcaaacatggtaggcgaaggcggtgatcctaataacagatcagtctttgaatgggcacatgacaggggccttgtctgtcagacgaacgtaacttgcttcttgaggaacgataacagtgggaaaccatacgaagtgaatcagacattgagggagaaacagttgtatacgaagaatgattccttgaacaacaccgattttggaactacctcggatgggcctggcgatgcgtacattggacatttggactatggtggtggagtggagtgtgatgcaatcttcccagactggggtcgaccagacgtggctcaatggtggggagaaaactacaagaagctgttcagcattggtctcgatttcgtgtggcaggatatgacggtacctgcgatgatgccgcaccgactcggtgatgctgtcaacaaaaattccggtagttcggcgccgggctggccgaatgagaacgatccatccaacggacgatacaactggaaatcttatcatccgcaagtgctcgtgaccgacatgcgctatggtgcagagtatggaagggaaccgatggtgtctcaacgcaacattcacgcctacactctttgtgaatctaccagacgggagggaattgtgggaaacgcagacagtttgaccaagttccgccgcagttacatcatcagtcgaggaggttacatcggtaaccagcatttcggagggatgtgggttggggacaacagtgccacagaatcctacctccaaatgatgttggcgaacattatcaacatgaatatgtcgtgcctcccgctagttggctctgatattggcgggttcacccagtacaatgatgcgggcgacccaacccccgaggatttgatggtaagattcgtgcaggctggctgtctgctaccgtggttcagaaaccactatgacaggtggattgagtccaagaagcacgggaagaaataccaggagttatacatgtacccggggcaaaaggatacgttgaagaagttcgttgaattccgctaccgctggcaggaggttttgtacacagccatgtaccaaaatgctaccactggagagccgatcatcaaggcggcgcccatgtacaacaacgacgtcaacgtgtataaatcgcagaatgatcatttccttctcggtggacatgacggctatcgtattctctgcgcacctgttgtgcgcgaaaatgcgacaagtcgcgaagtgtacctgcctgtgtatagcaagtggttcaaattcggaccggactttgacactaagcccttggaaaatgagattcaaggaggtcagacgctttataattacgctgcaccgctgaacgattcgccgatatttgtgagggaagggactattcttccgacacggtacacgctggacggtgtgaacaaatctatcaacacgtacacagacaatgatccgcttgtatttgagctgttccctctcgaaaacaaccaggcgcatggcttgttctatcatgatgatggcggtgtcaccaccaacgctgaagactttggcaagtattctgtgatcagtgtgaaggccgcgcaggaaggttctcaaatgagtgtcaagtttgacaatgaagtttatgaacaccaatggggagcatcgttctatgttcgtgttcgtaatatgggtgctccgtctaacatcaacgtatcttctcagattggtcaacaggacatgcaacagagctccgtgagttccagggcgcaaatgttcactagtgctaacgatggcgagtactgggttgaccagagcacgaactcgttgtggctcaagttgcctggtgcagttatccaagacgctgcgatcactgttcgttgagcgatatatccagttgtggagtttaatagtgtgttcagaagcgtgactggtaaccgcgatggtggtatcagtacatgttagtacaaataaacatcgtgctctatattgaggrcgacgtaatgcgttgttgttt

SEQ ID NO: 17 - protein, 163 aa, Peziza ostracoderma
FVWQDMTTPAIHTSYGDMKGFPTRLLVSSDGEPTSQIAPKEVLAIENWALFSYNLHKATFRGLDRLPSRSGKRNFILGRGSFSGAHRYAGLWTGDNASTWDFWRITVSQVLSVGLNGVSIAGSDMGGFEPAVGADGQEEKYCSPELLIRWYSGSVLLPWLRNH

SEQ ID NO: 18 - DNA, 557 bp, Peziza ostracoderma
ttcgtgtggcaagatatgaccaccccggccatccatacatcatacggtgacatgaaagggtaagtaccgccgaggcatcttctaacatcgtaaagctatgctcgcctactaatatgaaatcccaggtttccaactcgactcttggtgagttcagacggtgagccaacgtcacaaatagcaccaaaagaggtactcgctatcgagaactgggcccttttttcgtataatctccacaaagctactttccgtgggcttgatcgacttccatccagatccggaaaacgcaatttcatcttgggtcgtggaagtttttcgggcgcgcatcgttatgcgggattgtggacaggagacaatgccagcacatgggacttctggagaattacagtctcacaagtcctttccgtcggacttaacggtgtaagcattgcaggttctgatatgggtggttttgaaccggcggttggtgcggatggtcaggaggagaaatattgcagtccagagctccttattcgatggtattcaggttcggtcctcttgccatggcttagaaaccacta

SEQ ID NO: 19 - protein, 1025 aa, Trichodesmium erythraeum
MPSEIFKSDRLYKFIKVEEYFDHYQEWDKLGPAKNPVFDGKYTLSLTFDKAGGGECSMLLEITQNDVLRMRFNPNKKLAGDYARGTRVDNDETDEEIKRNNQRTVTYRQVKDIKLGDQVILTAKSNYNQVGRNTPFNMEVVITLNPFGIKVFNKSESNSEYANIPVWETANTSIYYTANGTDDYAIIQSVKKSADAKYIGFGEQGGTKLSKNMDQLNYFNFDNMRYRQVYNRGPLDNREPLYHSEPFFYEFNGVPGSDNVNAVLVDNPSQVFMDIGYSNSGRYMFGTRFGDLDYYVFFGEDPKNILDSYTAVIGRPELKPRYALGYHQGCYGYEKRSDLEWVVARYRDWGIPIDGLAVDVDLQANYRTFTININNFWEPDKMFDNLRKQGIKCCTNITPVISSQDKLGKTDYDYSTYVEGKNNNYFVVDKRYDPYNPISKEYQIYNGGIEDRSNKDDNSDPEGFDSSEPYIGEVYYGKDANGKELGSPGHYPDLGRQEVREWWGKQYQYLYEMGLEFVWQDMTTPAIRDFRGDMKGFPFRLYVTDDFYPSDVKLTPALKVWNLYSYNLHKATYEGLNNLYKLSKGLEWRENKRNYIIGRGSFSGSHRYSGLWTGDNSSEWAFLQMNISQVLSLGMNALAVTGQDIGGFEQEYGNDKQQWASPELVIRWTAAGAFLPWFRNHYVRKGRKEFQEPFQYIEWFETWNKPIPEPQDLYRMVPEICKHYIELRYRLMQLFYDTLFENTLDGLPICRPLFLNDPQDKSLYNDKDEFLNNEFFVGKDFLVAPVLLPQSETNGGKRDIYLPKPSYWYNFVNNVMPLNNALEGGTTIRDFDANINTRDQHINFIVPIYVRSAAIIPTIELEQYVGEKNAKGEKNPITLNIYPDYQKENGGEYHMYLDDGESRSSAPKSQVDDPKANDEYREILITNKYTGEKTREIKVNRVHDGYTPMEDFFFVAILHDPTEQKGEHGPLQEVTMEGKPQQMVSDNGALWGSPNNAWYYNADINISFIKVFDNMPKTTIMLGYV

SEQ ID NO: 20 - DNA, 3078 bp, Trichodesmium erythraeum
atgccgagtgaaatttttaaaagtgatcgcctctataaatttatcaaagtcgaagaatacttcgatcactaccaagaatgggataaactgggacctgcaaaaaatcctgtgttcgacgggaagtacactttaagccttacctttgataaggccggcggaggtgagtgctcaatgttgcttgagatcactcagaacgatgtcttacggatgcgctttaaccccaacaaaaaattagcaggggattatgcaaggggcactcgcgtggataatgatgaaacagatgaagagatcaaaaggaataatcaacgtactgtaacttacagacaagttaaggatattaagttaggagatcaggttatattgacggcaaaaagcaattataaccaggttggtcgtaatacacccttcaatatggaagtagtgatcactttaaatccatttggtatcaaagtattcaacaagtccgaatcgaattcagagtatgccaatattcctgtctgggaaaccgcaaacacttcaatttactataccgctaatggcacagatgattatgccatcattcagtcggtcaagaagtcggccgatgccaaatatattggttttggggaacaggggggaactaaactcagcaaaaatatggatcagttgaattatttcaactttgataatatgcgctatcgacaggtctataatcgtggacctttagataaccgcgaacctctttaccactctgagcctttcttttatgagttcaatggcgttcctggtagcgacaatgttaatgcagttctagtggataacccaagtcaagtattcatggacataggctatagtaattctggtcgctatatgtttgggactcgctttggtgatcttgactattatgtgttttttggagaagacccgaagaatattttagatagctatacggccgtgatcggtcgcccagagttgaaaccgcgctatgctttgggatatcaccaaggttgctatggatacgaaaagcggagtgacttagaatgggttgtggcgcgatatcgggattggggtatacccattgatggtctcgctgttgatgttgacttacaagcaaattatcggacttttactatcaacatcaataatttctgggaaccagataaaatgtttgataacttgcgaaagcaggggataaaatgctgtacgaacattactcccgtgatcagcagtcaagataagcttggcaagacagattatgactactcaacctacgttgagggaaagaataacaactactttgttgttgataagcgctacgatccatataatccaattagtaaagagtatcagatctataatgggggtattgaagatcgttccaacaaagatgataattccgatcccgaagggtttgatagtagtgagccttacattggcgaagtatattacggcaaggatgccaatggtaaagagctgggaagccctggtcattatcctgatttaggccgtcaagaagtacgcgaatggtggggcaagcaatatcaatatctgtatgaaatgggactagaatttgtctggcaggatatgactaccccagctatccgtgacttccgtggggatatgaaaggtttccccttccgtctgtatgtgactgacgacttctatccctcggatgtcaaactaaccccagctctgaaagtttggaatttgtattcatacaatttgcacaaagccacctacgaaggactcaataacctgtacaaactatccaaaggactagagtggcgcgaaaacaagcgcaactatatcatcggacgagggagtttttctggttctcacagatattctggtttatggactggggataactcttccgaatgggcttttcttcagatgaatatctctcaggttctgtctctaggcatgaacgctttggcagttaccgggcaagatatcggaggtttcgagcaagaatatggtaacgacaaacagcagtgggcgagtccggaactggtaattcgctggactgctgcaggtgccttcttaccatggttccgtaaccactatgtccgtaagggtcgtaaagaattccaagaaccattccaatatatagagtggtttgagacatggaacaagccaattcccgagccccaggacttgtacaggatggtgccagagatttgtaagcattatattgaattgcgctaccgtctgatgcagttattctacgacacattgtttgagaacactctggatggtctgcctatctgtcgtcctttgttcctcaacgatccccaggacaagtctctttacaatgataaagacgaatttttaaacaacgaatttttcgtaggcaaggattttctcgttgctccggtattgcttcctcagagtgaaaccaatggtggcaagcgggatatctatttacctaagcctagctactggtacaactttgtcaacaacgttatgcctcttaataatgctttggaagggggaacaacgatacgagattttgatgctaatatcaatactcgtgaccagcacattaactttattgtacccatctacgttcgctcggcagcaattattcccacaattgagttggaacaatatgtgggagaaaagaacgccaaaggagaaaaaaatcctattaccctgaatatctatccagactatcagaaggagaatggaggagaataccacatgtacttggatgatggggagagccgttcttctgccccgaaatcacaagtggacgatcccaaggcaaacgacgaatatcgcgagattttgattactaataaatacactggggaaaagaccagagagattaaagttaatcgtgttcacgatggatatacgcctatggaagactttttctttgtggcaatattgcacgatcctactgaacaaaagggagaacacggccctttgcaggaggtgactatggaaggtaaaccgcaacagatggttagtgacaatggtgccctctggggtagccctaacaacgcatggtactataatgcagacatcaatatcagttttatcaaggtgtttgataatatgcctaagacaactatcatgttaggttatgtctaa

SEQ ID NO: 21 - protein, 1100 aa, Artificial Sequence
  Description of Artificial Sequence Consensus sequence
MFXTLXFVAPSALGAXTFXXXXIXXSXIXIXSXXPAVXXAXRKSXXXNVSMTALXDKXTAXXXXXDNPDXIXYXXYDYVXVXXFXPLSNTNWFAAGSSTPGXIXDWTATMNVXFDRIDNPSXTXXXPVQVQVTSYXNNXFRVRFNPDGPIRDVXRGPILXQQLXWIRXQEXSXGXXPXMXFTXEGFLXFETKDLXVIIYGNXKXRVTRKXXXXXIMENXEXXXXSXGNKCRGLMFVDRLYGXAIASVNXNFXXDXXRXEXFYGAGEVNCXYXDXXXXXXXYILERTGIAMTNYNYDNLNYNQPDLIPPGHDXXSDPDYYIPMYFAAPWVIVXGCAGXTSDQYSYGWFMDNVSQSYMNTGDTAWNXGQEDLAYMGAQCGPFDQHFVYGAGDGLEDVVTAFSYLQGKEFENQXLNKRSVMPPKYVFGFFQGVFGATSLLRDNLPAGENNISVEEIVEGYQNNNFPFEGLAVDVDMQDDLRVFTTKPEFWTANKVGXGGDPNNKSVFEWAHDRGLVCQTNVTCFLRNDNXGXPYEVNQTLREKQLYTKNDSLXNTDFGTTXDGPSDAYIGHLDYGGGVECDALFPDWGRPDVAQWWGDNYKKLFSIGLDFVWQDMTVPAMMPHKLGDAVNTRSXXXXXGWPNEXDPSNGRYNWKSYHPQVLVTDMRYEXHGXREPMVTQRNIHAYTLCESTRKEGIVGNADSLTKFRRSYIISRGGYIGNQHFGGMWVGDNSSTEXYLQMMIANIINMNMSCLPLVGSDIGGFTXYDXRNXXXPCPXDLMVRFVQAGCLLPWFRNHYXRXIEXKXXGKXYQELYMYXXXXXTLRKFVEFRYRWQEVLYTAMYQNAXXGXPIIKAAXMYNNDXNVXXAQXDHFLLGGHDGYRILCAPVVXENXTXRELYLPVXTXWYKFGPDFDTKXLEXXIXGGXXIXNYXXPXXDSPIFVREGXILPTRYTLXGXNKSINTYTDXDPLVFEVFPLXNNXAXGMXYXDDGGVTTXAEDXGKFSVIXVXAXXXGXXXTIXFXXDXYXYXFXGXFYVRVRXXXXXSXIXVSSXXGXXDMXXSSXXSRAXLFXXGXXGEYWXDQXTXSLWLKLPXXVLXDAXITIX

SEQ ID NO: 22 - protein, 1100 aa, Artificial Sequence
  Description of Artificial Sequence Consensus sequence
MAGXSDPLNFCKAEDYYAXAXXWXGPQKIIXXDXTPPXXTKXPKXWHAVNLXFDDGTLXVVQFIRPCVWRVRYDPXXKTSDEYGDENTRTIVQDYMSTLXXXLDXFRGLTWXSXXEDSGDFFTFXSXVTAVDXSERTRNKVGDGLKIHLWKXPFRIQVVRXLTPLXDPFPIPNVAXAXARVADKVVWQTSPKTFRKNLHPQHKMLKDTVLDIIKPGHGEYVGWGEMGGIXFMKEPTFMNYFNFDNMQYQQVYAQGALDSREPLYHSDPFYLDVNSNPEHKNITATFIDNYSQIAIDFGKTNSGYIKLGTRYGGIDCYGISADTVPEIVRLYTGLVGRSKLKPRYILGAHQACYGYQQESDLHAVVQQYRDXKFPLDGIHVDVDXQDXFRTFTTNPXTFPNPKEMFTNLRNNGIKCSTNITPVISIXXRXXGYSTLXEGXDKKYFIMDDRYTEGTSGXXXXVRYXFYGGGNXVEVXPNDVXARPDFXDNYDFPXNFNXKXYPYHGGVSYGYGNGSXGFYPDLNRXEVRIWWGLQYXYLFXMGLEFVWQDMTTPAIHTSYGDMKGLPTRLLVTSDSVTNASEKXXXKLAIESWALYSYNLHKATFHGLXRLESRKNKRNFILGRGSYAGAYRFAGLWTGDNASTWEFWKISVSQVLSLGLNGVCIAGSDTGGFEPARXAXGXEEKYCSPELLIRWYTGSFLLPWLRNHYVKKDRKWFQEPYAYPKHLETHPELADQAWLYKSVLEICRYWVELRYSLIQLLYDCMFQNVVDGMPIXRSMLLTDTEDTTFFNESQKFLDNQYMAGDDILVAPILHSRXEIPGENRDVYLPLFHTWYPSNLRPWDDQGVALGNPVEGGSVINYTARIVAPEDYNLFHXVVPVYIREGAIIPQIXVRQWXGXGGXNXIKFNIYPGKDKEYXTYLDDGVSRDSAPDDLPQYKEXHEQAKVEGXDIXKQIAXXXGXXXXXFXXSGXDXEAKGYHRKVAIXQXSKDKTRTVTIEPKHNGYHRKVAIXQXSKDKTRTVTIEPKHNGYDPSKEVGXYYTIILWYAPGFDGSIVDVSXXTVNIEGGVEXXIFKNSXLHTVVIXVKEVIGTTKSVKITCTXA

SEQ ID NO: 23 - DNA, 30 bp, Artificial Sequence
  Description of Artificial Sequence Primer
tgctctagagaacaatggcttcctctatgc

SEQ ID NO: 24 - DNA, 40 bp, Artificial Sequence
  Description of Artificial Sequence Primer
gtttgtcggacaatgcggtcatgcagttaactcttccgcc

SEQ ID NO: 25 - DNA, 38 bp, Artificial Sequence
  Description of Artificial Sequence Primer
tgctctagacaacaatgttttcaacccttgcgtttgtc

SEQ ID NO: 26 - DNA, 21 bp, Artificial Sequence
  Description of Artificial Sequence Primer
gtatgacgtgacctgaacctg

SEQ ID NO: 27 - DNA, 30 bp, Artificial Sequence; identical to SEQ ID NO: 23
  Description of Artificial Sequence Primer

SEQ ID NO: 28 - DNA, 21 bp, Artificial Sequence
  Description of Artificial Sequence Primer
gtatgacgtgacctgaacctg

SEQ ID NO: 29 - DNA, 24 bp, Artificial Sequence
  Description of Artificial Sequence Primer
agcggataacaatttcacacagga

SEQ ID NO: 30 - DNA, 21 bp, Artificial Sequence
  Description of Artificial Sequence Primer
gtatgacgtgacctgaacctg

SEQ ID NO: 31 - protein, 320 aa, Gracilariopsis lemaneiformis
MTNYNYDNLNYNQPDVVPPGYPDHPNYYIPMYYAAPWLVVQGCAGTSKQYSYGWFMDNVSQSYMNTGDTAWNCGQENLAYMGAQYGPFDQHFVYGDGDGLEDVVKAFSFLQGKEFEDKKLNKRSVMPPKYVFGFFQGVFGALSLLKQNLPAGENNISVQEIVEGYQDNDYPFEGLAVDVDMQDDLRVFTTKPEYWSANMVGEGGDPNNRSVFEWAHDRGLVCQTNVTCFLRNDNSGKPYEVNQTLREKQLYTKNDSLNNTDFGTTSDGPGDAYIGHLDYGGGVECDAIFPDWGRPDVAQWWGENYKKLFSIGLDFVWQDM

(19) United States
(12) Patent Application Publication
(10) Pub. No.: US 2005/0044589 A1
(43) Pub. Date: Feb. 24, 2005

(54) Process for preparing an anti-oxidant in a plant by transformation with glucan lyase DNA

(76) Inventors: Aksel Buchter-Larsen, Copenhagen (DK); Jan Marcussen, Copenhagen (DK)

Correspondence Address: THOMAS J. KOWALSKI, Esq.;c/o FROMMER LAWRENCE &, HAUG LLP, 745 Fifth Avenue, New York, NY, 10151, US

(21) Appl. No.: 10/879,638

(22) Filed: Jun. 29, 2004

Related U.S. Application Data
(63) Continuation-in-part of application No. 09/423,126, filed on Nov. 5, 1999, now abandoned.
Application No. 09/423,126 is a continuation of application No. PCT/IB98/00708, filed on May 6, 1998.

(30) Foreign Application Priority Data
May 6, 1997 (GB) 9709161.5

(51) Int. Cl. A01H 1/00; C12N 15/82; C12N 5/04; C12N 5/10

(52) U.S. Cl. 800/284; 434/161; 435/468; 435/419

(57) ABSTRACT

A process of preparing an anti-oxidant in a plant is described. The process comprises transforming a plant with a nucleic acid encoding glucan lyase, thereby producing the anti-oxidant, anhydrofructose, in situ.

### CROSS-REFERENCE TO RELATED APPLICATIONS

This application is a continuation-in-part of U.S. application Ser. No. 09/423,126, filed on Nov. 5, 1999 as a continuation of International Application No. PCT/IB98/00708, filed on May 6, 1998, designating the U.S., published as WO 98/50532 on Nov. 12, 1998, and claiming priority to GB application Ser. No. 9709161.5, filed on May 6, 1997.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a process of preparing an anti-oxidant in situ in a plant.

### BACKGROUND OF THE INVENTION

An anti-oxidant prevents, inhibits or reduces the oxidation rate of an oxidisable medium. In particular, anti-oxidants are used for the preservation of food, especially when the food is or comprises a fat. Typical chemical anti-oxidants include aromatic amines, substituted phenols and sulphur compounds. Examples of anti-oxidants for food products are polyvinylpolypyrrolidone, dithiothreitol, sulphur dioxide, synthetic gamma.-tocopherol, .delta.-tocopherol, L-ascorbic acid, sodium L-ascorbate, calcium L-ascorbate, ascorbyl palmitate, propyl gallate, octyl gallate, dodecyl gallate, lecithin, diphenylamine ethoxyquin and butylated hydroxytoluene. Two commonly used anti-oxidants are GRINDOX 142 (obtained from Danisco A/S) and GRINDOX 1029 (obtained from Danisco A/S).

Typically, anti-oxidants are added as “chemical” additives to foodstuffs, such as beverages. For example, anti-oxidants are used in the preparation of alcoholic beverages such as beer, cider, ale etc. In particular, there is a wide spread use of anti-oxidants in the preparation of wine. In this regard, Butzke and Bisson in Agro-Food-Industry Hi-Tech (July/August 1996 pages 26-30) present a review of wine manufacture.

According to Butzke and Bisson (ibid):
Wine is the product of the natural fermentation of grape must or juice. In the case of red wine, the skins are present during the initial fermentation to allow extraction of pigment and important flavour and aroma constituents from the skin. The term “must” refers to the crushed whole grapes. In the case of white wine production, skins are removed prior to fermentation and only the juice is retained and processed . . .
Grapes are harvested and brought directly to the winery from the field. The grapes are then crushed at the winery and the must either transferred to a tank for fermentation (red wine) or pressed to separate juice from the skin and seeds (white wine). In this latter case, the juice is then transferred to a tank for fermentation. The tanks may either be inoculated with a commercial wine strain of Saccharomyces or allowed to undergo a natural or uninoculated fermentation. In a natural fermentation, Saccharomyces cells are greatly outnumbered by wild (non-Saccharomyces) yeast and bacteria at the beginning of fermentation. By the end of the fermentation Saccharomyces is the dominant and most often only organism isolateable. Inoculation with a commercial wine strain or with fermenting juice or must changes the initial ratio of the numbers of different microorganisms, allowing Saccharomyces to dominate the fermentation much earlier.
The metabolic activity of microorganisms in wine results in the production of aroma and flavour compounds some of which are highly objectionable to the consumer and all of which are distinct from the compounds responsible for the varietal character of the wine . . . Sulphur dioxide addition prevents chemical oxidation reactions and in this sense is an important stabilizer of the natural grape aroma and flavour. It may be added to the must or juice to preserve flavour, not necessarily as an antimicrobial agent. However, its antimicrobial activity must be considered when choosing a strain to be genetically modified for wine production.

Hence, potentially harmful chemicals—such as sulphur dioxide—are used in wine manufacture to prevent chemical oxidation reactions and stabilize natural grape aromas and flavors. The addition of chemical additives to foodstuffs is disadvantageous.

As an alternative to chemical anti-oxidants, “natural” anti-oxidants, for example anhydrofructose, can be produced in microorganisms, such as bacteria, yeast and fungi. Some researchers have focused on large-scale production of anhydrofructose using microorganisms (Yu et al. U.S. Pat. No. 6,013,504; Yu et al. WO 95/10618).

The present invention is predicated upon the realization that anti-oxidants, for example anhydrofructose, could be prepared in plants in situ. Thus, the present invention overcomes the problem of the unwanted addition of potentially harmful chemicals and chemical antioxidants to foodstuffs. A further advantage of providing an antioxidant in a foodstuff is that antioxidants are often taken as nutritional supplements. Thus the production of a foodstuff with in situ above-normal levels of an antioxidant, such as anhydrofructose, may circumvent the need for additional nutritional supplements of antioxidants. An additional advantage of the invention is to assist transformation of a plant, e.g. a grape, transformed with a nucleotide sequence encoding glucan lyase, which, in situ, produces the antioxidant anhydrofructose.

### SUMMARY OF THE INVENTION

The present invention seeks to overcome any problems associated with the prior art methods of preparing foodstuffs with antioxidants.

According to a first aspect of the present invention there is provided a process of preparing a medium that comprises an anti-oxidant and at least one other component, the process comprising preparing in situ in the medium the anti-oxidant; and wherein the anti-oxidant is prepared from a glucan by use of recombinant DNA techniques.

According to a second aspect of the present invention there is provided a process of preparing a medium that comprises an anti-oxidant and at least one other component, the process comprising preparing in situ in the medium the anti-oxidant; and wherein the anti-oxidant is prepared by use of a recombinant glucan lyase.

According to a third aspect of the present invention there is provided a medium prepared by the process according to the present invention.

Other aspects of the present invention include:

Use of anhydrofructose as an anti-oxidant for a medium comprising at least one other component, wherein the anhydrofructose is prepared in situ in the medium.

Use of anhydrofructose as a means for imparting or improving stress tolerance in a plant, wherein the anhydrofructose is prepared in situ in the plant.

Use of anhydrofructose as a means for imparting or improving the transformation of a grape, wherein the anhydrofructose is prepared in situ in the grape.

Use of anhydrofructose as a means for increasing antioxidant levels in a foodstuff (preferably a fruit or vegetable, more preferably a fresh fruit or a fresh vegetable), wherein the anhydrofructose is prepared in situ in the foodstuff.

Use of anhydrofructose as a pharmaceutical in a foodstuff, wherein the anhydrofructose is prepared in situ in the foodstuff.

A method of administering a foodstuff comprising anhydrofructose, wherein the anhydrofructose is in a pharmaceutically acceptable amount and acts as a pharmaceutical; and wherein the anhydrofructose has been prepared in situ in the foodstuff.

Use of anhydrofructose as a nutraceutical in a foodstuff, wherein the anhydrofructose is prepared in situ in the foodstuff.

A method of administering a foodstuff comprising anhydrofructose, wherein the anhydrofructose is in a nutraceutically acceptable amount and acts as a nutraceutical; and wherein the anhydrofructose has been prepared in situ in the foodstuff.

Use of glucan lyase as a means for imparting or improving stress tolerance in a plant, wherein the glucan lyase is prepared in situ in the plant.

Use of glucan lyase as a means for imparting or improving the transformation of a grape, wherein the glucan lyase is prepared in situ in the grape.

Use of glucan lyase as a means for increasing antioxidant levels in a foodstuff (preferably a fruit or vegetable, more preferably a fresh fruit or a fresh vegetable), wherein the glucan lyase is prepared in situ in the foodstuff.

Use of glucan lyase in the preparation of a pharmaceutical in a foodstuff, wherein the glucan lyase is prepared in situ in the foodstuff.

A method of administering a foodstuff comprising an antioxidant, wherein the antioxidant is in a pharmaceutically acceptable amount and acts as a pharmaceutical; and wherein the antioxidant has been prepared in situ in the foodstuff from a glucan lyase.

Use of glucan lyase in the preparation of a nutraceutical in a foodstuff, wherein the glucan lyase is prepared in situ in the foodstuff.

A method of administering a foodstuff comprising an antioxidant, wherein the antioxidant is in a nutraceutically acceptable amount and acts as a nutraceutical; and wherein the antioxidant has been prepared in situ in the foodstuff from a glucan lyase.

Use of a nucleotide sequence coding for a glucan lyase as a means for imparting or improving stress tolerance in a plant, wherein the nucleotide sequence is expressed in situ in the plant.

Use of a nucleotide sequence coding for a glucan lyase as a means for imparting or improving the transformation of a grape, wherein the nucleotide sequence is expressed in situ in the grape.

Use of a nucleotide sequence coding for a glucan lyase as a means for increasing antioxidant levels in a foodstuff (preferably a fruit or vegetable, more preferably a fresh fruit or a fresh vegetable), wherein the nucleotide sequence is expressed in situ in the foodstuff.

Use of a nucleotide sequence coding for a glucan lyase as a means for creating a pharmaceutical in a foodstuff, wherein the nucleotide sequence is expressed in situ in the foodstuff.

A method of administering a foodstuff comprising an antioxidant, wherein the antioxidant is in a pharmaceutically acceptable amount and acts as a pharmaceutical; and wherein the antioxidant has been prepared in situ in the foodstuff by means of a nucleotide sequence coding for a glucan lyase.

Use of a nucleotide sequence coding for a glucan lyase as a means for creating a nutraceutical in a foodstuff, wherein the nucleotide sequence is expressed in situ in the foodstuff.

A method of administering a foodstuff comprising an antioxidant, wherein the antioxidant is in a nutraceutically acceptable amount and acts as a nutraceutical; and wherein the antioxidant has been prepared in situ in the foodstuff by means of a nucleotide sequence coding for a glucan lyase.

A method for increasing the degradation of an α-1,4-glucan substrate in a plant or part thereof, the method comprising introducing a nucleic acid encoding an enzyme selected from the group consisting of α-glucosidase and α-1,4-glucan lyase into the plant or part thereof, wherein the enzyme is expressed and acts on the glucan substrate to yield increased degradation of the glucan substrate in the plant or part thereof. The term “nutraceutical” means a compound that is capable of acting as a nutrient (i.e. it is suitable for, for example, oral administration) as well as being capable of exhibiting a pharmaceutical effect and/or cosmetic effect.

The present invention is also believed to be advantageous as it provides a means of improving stress tolerance of plants.

The present invention is also advantageous as it provides a means for viably transforming grape.

The present invention is further advantageous in that it enables the levels of antioxidants in foodstuffs to be elevated. This may have beneficial health implications. In this regard, recent reports (e.g. Biotechnology Newswatch Apr. 21 1997 “Potent Antioxidants, as strong as those in fruit, found in coffee” by Marjorie Shaffer) suggest that antioxidants have a pharmaceutical benefit, for example in preventing or suppressing cancer formation.

### BRIEF DESCRIPTION OF THE DRAWINGS

Various preferred features and embodiments of the present invention will now be described in more detail by way of non-limiting example and with reference to the accompanying drawings, in which:

FIGS. 1A and 1B show the amino acid sequence alignment of glucan lyases GLq1 (SEQ ID NO: 1), GLq2 (SEQ ID NO: 2), GLq3 (SEQ ID NO: 31), GLs1 (SEQ ID NO: 5), GLs2 (SEQ ID NO: 6) and GLa1 (SEQ ID NO: 13), isolated from red algae, and the consensus sequence (SEQ ID NO: 21).

FIG. 2 shows the amino acid sequence alignment of glucan lyases GLmc (SEQ ID NO: 3), GLmv (SEQ ID NO: 4) and GLpo (SEQ ID NO: 17), isolated from fungi, and the consensus sequence (SEQ ID NO: 22).

FIG. 3 shows wild type (wt) and transgenic potato leaves stained with Lugol solution (iodine/potassium iodide) to qualitatively reveal the starch content. No starch was detected in transgenic lines 11.1, 14.1 or 14.3, all of which contain an active glucan lyase gene. In contrast, both wt plants and the transgenic line 8.1, in which no active glucan lyase was detected, clearly contain starch.

FIG. 4 shows a quantitative determination of anhydro fructose in wt (red) and transgenic line 11.1 (blue) extracts analyzed by reverse phase HPLC.

FIG. 5 shows wt and transgenic Arabidopsis thaliana stained with Lugol solution to qualitatively reveal the starch content. No starch was detected in transgenic line SR2, which contains an active glucan lyase gene. In contrast, the wt plant, in which there is no glucan lyase gene, clearly contains starch.

### DETAILED DESCRIPTION

According to the present invention, there is provided a method of preparing in situ in an oxidisable medium an anti-oxidant. In a preferred embodiment, the anti-oxidant is anhydrofructose, more preferably 1,5-anhydro-D-fructose. 1,5-anhydro-D-fructose has been chemically synthesised (Lichtenthaler in Tetrahedron Letters Vol 21 pp 1429-1432), and is further discussed in WO 95/10616, WO 95/10618 and GB-B-2294048.

The main advantages of using 1,5-anhydro-D-fructose as an anti-oxidant are that it is a natural product, it is non-metabolisable, it is easy to manufacture, it is water-soluble, and it is generally non-toxic.

The in situ preparation of anti-oxidants is particularly advantageous in that less, or even no, additional anti-oxidants need be added to the medium, such as a food product. An anti-oxidant that is prepared in situ in the medium can be used as the main anti-oxidant in the medium.

In a preferred embodiment, the anti-oxidant is prepared in a medium that is or is used to make a foodstuff, such as a beverage. The beverage can be an alcoholic beverage, in particular, wine. It is preferred that the medium is a plant, advantageously a grape plant, or a part thereof. The medium can be a cereal or fruit.

In the alternative, the medium may be for use in polymer chemistry. In this regard, the in situ generated anti-oxidants could therefore act as oxygen scavengers during, for example, the synthesis of polymers, such as the synthesis of biodegradable plastic.

The term “in situ in the medium” as used herein includes the anti-oxidant being prepared by action of a recombinant enzyme expressed by the component on a glucan—which glucan is a substrate for the enzyme. The term also includes the anti-oxidant being prepared by action of a recombinant enzyme expressed by the component on a glucan—which glucan is a substrate for the enzyme—within the component and the subsequent generation of the anti-oxidant. The term also includes the recombinant enzyme being expressed by the component and then being released into the medium, which enzyme acts on a glucan—which glucan is a substrate for the enzyme—present in the medium to form the anti-oxidant in the medium. The term also covers the presence or addition of another component to the medium, which component then expresses a recombinant nucleotide sequence which results in exposure of part or all of the medium to an anti-oxidant, which anti-oxidant may be a recombinant enzyme or a recombinant protein expressed and released by the other component, or it may be a product of a glucan—which glucan is a substrate for the enzyme—within the medium that has been exposed to the recombinant enzyme or the recombinant protein.

General in situ preparation of antioxidants in plants has been previously reviewed by Badiani et al in Agro-Food-Industry Hi-Tech (March/April 1996 pages 21-26). It is to be noted, however, that this review does not mention preparing in situ antioxidants from a glucan, let alone by use of a recombinant glucan lyase.

The term “by use of recombinant DNA techniques” as used herein includes the anti-oxidant being any obtained by use of a recombinant enzyme or a recombinant protein, which enzyme or protein acts on the glucan. The term also includes the anti-oxidant being any obtained by use of an enzyme or protein, which enzyme or protein acts on a recombinant glucan.

The term “starch” in relation to the present invention includes native starch, degraded starch, modified starch, including its components amylose and amylopectin, and the glucose units thereof.

The terms “variant”, “homologue” or “fragment” in relation to the enzyme include any substitution of, variation of, modification of, replacement of, deletion of or addition of one (or more) amino acid from or to the sequence providing the resultant amino acid sequence has α-glucan lyase activity, preferably having at least the same activity of any one of the enzymes shown as SEQ ID NO: 1-6, 13-15, 17 or 19. In particular, the term “homologue” covers homology with respect to structure and/or function providing the resultant enzyme has α-glucan lyase activity. With respect to sequence homology, preferably there is at least 75%, more preferably at least 85%, more preferably at least 90% homology to any one of the sequences shown as SEQ ID NO: 1-6, 13-15, 17 or 19. More preferably there is at least 95%, more preferably at least 98%, homology to any one of the sequences shown as SEQ ID NO: 1-6, 13-15, 17 or 19.

The terms “variant”, “homologue” or “fragment” in relation to the nucleotide sequence coding for the enzyme include any substitution of, variation of, modification of, replacement of, deletion of or addition of one (or more) nucleic acid from or to the sequence providing the resultant nucleotide sequence codes for an enzyme having α-glucan lyase activity, preferably having at least the same activity of any one of the enzymes shown as SEQ ID NO: 1-6, 13-15, 17 or 19. In particular, the term “homologue” covers homology with respect to structure and/or function providing the resultant nucleotide sequence codes for an enzyme having α-glucan lyase activity. With respect to sequence homology, preferably there is at least 75%, more preferably at least 85%, more preferably at least 90% homology to any one of the sequences shown as SEQ ID NO: 7-12, 16, 18 or 20. More preferably there is at least 95%, more preferably at least 98%, homology to any one of the sequences shown as SEQ ID NO: 7-12, 16, 18 or 20.

As used herein, the term “sequence homology” can be equated with “sequence identity”.

The above terms are synonymous with allelic variations of the sequences.

The present invention also covers nucleotide sequences that can hybridise to the nucleotide sequence of the present invention.

The term “nucleotide” in relation to the present invention includes cDNA.

Glucan Lyases

Glucan lyases are enzymes, which produce 1,5-anhydro-D-fructose (anhydrofructose) from starch and related oligomers and polymers. In plants, starch biosynthesis takes place exclusively in plastids that are the sole location of starch synthases and starch branching enzymes (Preiss 1997, Manipulation of starch synthesis. In A Molecular Approach to Primary Metabolism in Plants (Quick, W. P. and Foyer, C. H., eds). London: Taylor and Francis, pp. 81-103).

In a preferred embodiment, the glucan is starch or a unit of starch and comprises α-1,4 links. Preferably, the glucan is a substrate for a recombinant enzyme such that contact of the glucan with the recombinant enzyme yields the anti-oxidant. The enzyme can be a glucan lyase, preferably an α-1,4-glucan lyase.

Some glucan lyase enzymes have high substrate specificity for maltose and maltosaccharides. Therefore, in an additional embodiment of the invention, the glucan is a maltose and/or a maltosaccharide substrate.

Preferably, the enzyme comprises or is one of the sequences shown as SEQ ID NO: 1-6, 13-15, 17 or 19, or a variant, homologue or fragment thereof. The enzyme can be encoded by a nucleotide sequence that comprises or is one of the sequences shown as SEQ ID NO: 7-12, 16, 18 or 20, or a variant, homologue or fragment thereof.

A summary of some glucan lyases and some of the nucleotide sequences encoding them, any of which is suitable for use in the present invention for producing 1,5-anhydro-D-fructose from starch, is shown in Table 1. A more detailed description of the glucan lyases can be found in WO 95/10616, WO 95/10618, GB-B-2294048, Yu (2004, Zuckerindustrie 129(10:26-30), Yu et al. (2004, Biochim. et Biophys. Acta 1672:120-129), Bojsen et al.(a) (1999, Plant Molec. Biol. 40:445-454) and Bojsen et al.(b) (1999, Biochim. et Biophys. Acta 1430:396-402). GLv1 is also discussed further in WO 94/09122. The designations for the enzymes and genes encoding them are discussed, for example, in Bojsen et al.(a), Bojsen et al.(b) and Yu. Accession numbers are provided in Bojsen et al.(b).

TABLE 1

| Present Appln. | WO 95/10616 | WO 95/10618 | GB 2 294 048 | Designation |
|---|---|---|---|---|
| SEQ ID NO: 1 | SEQ ID NO: 1 | SEQ ID NO: 1 | | GLq1 |
| SEQ ID NO: 2 | SEQ ID NO: 2 | SEQ ID NO: 2 | | GLq2 |
| SEQ ID NO: 3 | SEQ ID NO: 5 | | | GLmc (McAGLL1) |
| SEQ ID NO: 4 | SEQ ID NO: 6 | | | GLmv (MvAGLL1) |
| SEQ ID NO: 5 | | | SEQ ID NO: 3 | GLs1 |
| SEQ ID NO: 6 | | | SEQ ID NO: 4 | GLs2 |
| SEQ ID NO: 7 | SEQ ID NO: 3 | SEQ ID NO: 3 | | GLq1 (GlAgll1) |
| SEQ ID NO: 8 | SEQ ID NO: 4 | SEQ ID NO: 4 | | GLq2 (GlAgll2) |
| SEQ ID NO: 9 | SEQ ID NO: 7 | | | GLmc (Agll1; Mo.cos) |
| SEQ ID NO: 10 | SEQ ID NO: 8 | | | GLmv (Agll1; Mo.vul) |
| SEQ ID NO: 11 | | | SEQ ID NO: 1 | GLs1 (GlAgll4) |
| SEQ ID NO: 12 | | | SEQ ID NO: 2 | GLs2 (GlAgll5 |
| SEQ ID NO: 13 | | | | GLa1 |
| SEQ ID NO: 14 | | | | GLan |
| SEQ ID NO: 15 | | | | GLq3 |
| SEQ ID NO: 16 | | | | GLq3 (GlAgll3) |
| SEQ ID NO: 17 | | | | GLpo (PoAGLL1) |
| SEQ ID NO: 18 | | | | GLpo (Agll1; Pe.ost) |
| SEQ ID NO: 19 | | | | |
| SEQ ID NO: 20 | | | | |

Table 2 shows the sources of the glucan lyases listed in Table 1.

TABLE 2

| Sequence | Type | Source |
|---|---|---|
| SEQ ID NO: 1 | amino acid | Gracilariopsis lemaneiformis |
| | | subspecies from Qingdao (algal) |
| SEQ ID NO: 2 | amino acid | Gracilariopsis lemaneiformis |
| | | subspecies from Qingdao (algal) |
| SEQ ID NO: 3 | amino acid | Morchella costata (fungal) |
| SEQ ID NO: 4 | amino acid | Morchella vulgaris (fungal) |
| SEQ ID NO: 5 | amino acid | Gracilariopsis lemaneiformis |
| | | subspecies from Santa Cruz (algal) |
| SEQ ID NO: 6 | amino acid | Gracilariopsis lemaneiformis |
| | | subspecies from Santa Cruz (algal) |
| SEQ ID NO: 7 | nucleotide | Gracilariopsis lemaneiformis |
| | | subspecies from Qingdao (algal) |
| SEQ ID NO: 8 | nucleotide | Gracilariopsis lemaneiformis |
| | | subspecies from Qingdao (algal) |
| SEQ ID NO: 9 | nucleotide | Morchella costata (fungal) |
| SEQ ID NO: 10 | nucleotide | Morchella vulgaris (fungal) |
| SEQ ID NO: 11 | nucleotide | Gracilariopsis lemaneiformis |
| | | subspecies from Santa Cruz (algal) |
| SEQ ID NO: 12 | nucleotide | Gracilariopsis lemaneiformis |
| | | subspecies from Santa Cruz (algal) |
| SEQ ID NO: 13 | amino acid | Gracilariopsis lemaneiformis |
| | | subspecies from Araya Peninsula (algal) |
| SEQ ID NO: 14 | amino acid | Anthracobia melaloma (fungal) |
| SEQ ID NO: 15 | amino acid | Gracilariopsis lemaneiformis |
| | | subspecies from Qingdao (algal) |
| SEQ ID NO: 16 | nucleotide | Gracilariopsis lemaneiformis |
| | | subspecies from Qingdao (algal) |
| SEQ ID NO: 17 | amino acid | Peziza ostracoderma (fungal) |
| SEQ ID NO: 18 | nucleotide | Peziza ostracoderma (fungal) |
| SEQ ID NO: 19 | amino acid | Trichodesmium erythraeum (bacterial) |
| SEQ ID NO: 20 | nucleotide | Trichodesmium erythraeum (bacterial) |

Another glucan lyase, designated GLv1, was isolated from Gracilaria verrucosa from Araya Peninsula.

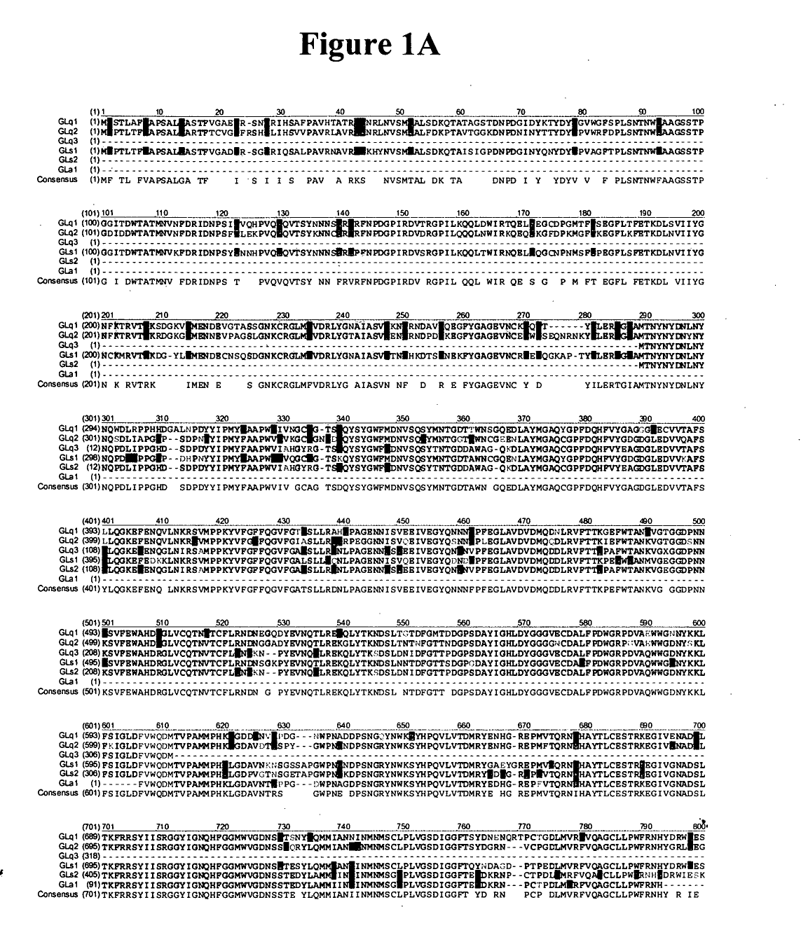

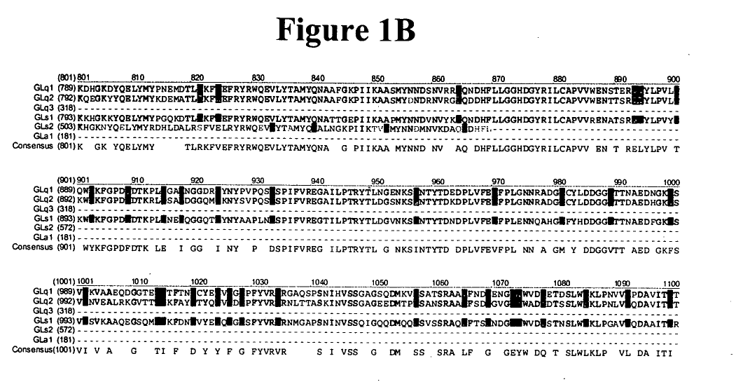

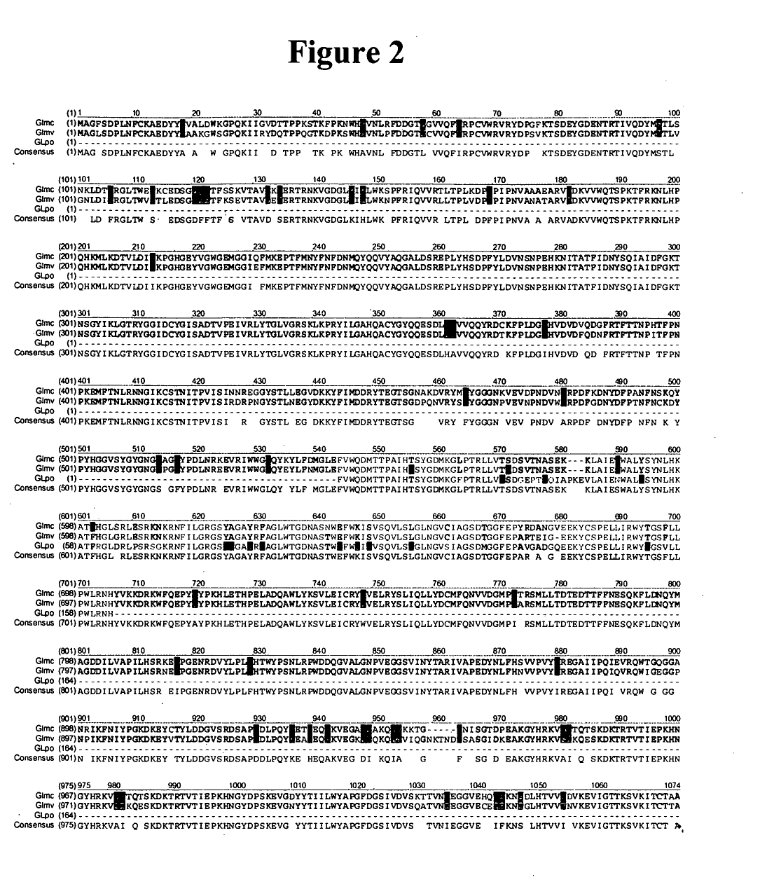

A sequence comparison of the glucan lyases discussed herein is shown in Table 3. Values are given as percent sequence identity between amino acid sequences. Sequence alignments are shown in FIGS. 1 and 2.

TABLE 3

| SEQ ID NO: | 1 | 2 | 3 | 4 | 5 | 6 | 13 | 14 | 16 | 17 |
|---|---|---|---|---|---|---|---|---|---|---|
| 1 | 100 | 76 | 26 | 40 | 73 | 76 | 82 | 28 | 83 | 24 |
| 2 | 76 | 100 | 25 | 25 | 70 | 75 | | | | |
| 3 | 26 | 25 | 100 | 85 | 24 | 25 | | 51 | | 76 |
| 4 | 40 | 25 | 85 | 100 | 24 | 26 | | | | |
| 5 | 73 | 70 | 24 | 24 | 100 | 80 | | | | |
| 6 | 76 | 75 | 25 | 26 | 80 | 100 | | | | |
| 13 | 82 | | | | | | 100 | | | |
| 14 | 28 | | 51 | | | | | 100 | | |
| 15 | 83 | | | | | | | | 100 | |
| 17 | 24 | | 76 | | | | | | | 100 |
| 19 | | | | 51 | | | | | | |

In spite of the fact that some of these enzymes have relatively low sequence identity with other members of the group, they are all members of the glucan lyase family and catalyze the preparation of anhydrofructose from an α-1,4-glucan based substrate. In addition, there is substantial identity between enzymes that were isolated from the same source. For example, the algal glucan lyases (SEQ ID NO: 1, 2, 5, 6, 13 and 15) share approximately 70-85% sequence identity with one another.

1,5-anhydro-D-fructose can be prepared by the enzymatic modification of substrates based on α-1,4-glucan by use of the enzyme α-1,4-glucan lyase. (See Yu et al. 1999, Biochimica et Biophysica Acta 1433:1-15.) A typical α-1,4-glucan based substrate is starch.

Today, starches have found wide uses in industry mainly because they are cheap raw materials. There are many references in the art to starch. For example, starch is discussed by Salisbury and Ross in Plant Physiology (Fourth Edition, 1991, Published by Wadsworth Publishing Company—especially section 11.7). In short, however, starch is one of the principal energy reserves of plants. It is often found in colourless plastids (amyloplasts), in storage tissue and in the stroma of chloroplasts in many plants. Starch is a polysaccharide carbohydrate. It comprises two main components: amylose and/or amylopectin. Both amylose and/or amylopectin consist of straight chains of α(1,4)-linked glucose units (i.e. glycosyl residues) but in addition amylopectin includes α (1,6) branched glucose units.

The recombinant nucleotide sequences coding for the enzyme may be cloned from sources such as a fungus, preferably Morchella costata or Morchella vulgaris, or from a fungally infected algae, preferably Gracilariopsis lemaneiformis, from algae alone, preferably Gracilariopsis lemaneiformis, and from cyanobacteria, preferably Trichodesmium erythraeum. It is likely that glucan lyase exists in organisms other than these, due to the wide occurrence in many organisms of anhydroglucitol, the reduced form of anhydrofructose. In further support of this belief, anhydrofructose has been identified in E. coli and mammals (Shiga et al. 1999, J. Biochem. 125:166-172; Suzuki et al. 1996, Eur. J. Biochem. 240:23-29). Glucan lyase may not be reported in numerous taxonomic groups because of low levels and/or lack of suitable detection and isolation methods.

In a preferred embodiment, the 1,5-anhydro-D-fructose is prepared in situ by treating an α-1,4-glucan with a recombinant α-1,4-glucan lyase, such as any one of those presented as SEQ ID NO: 1-6, 13-15, 17 or 19.

Detailed commentary on how to prepare the enzymes shown as sequences SEQ ID NO: 1-6 maybe found in the teachings of WO 95/10616, WO 95/10618 and GB-B-2294048. Likewise, detailed commentary on how to isolate and clone the nucleotide sequences SEQ ID NO: 7-12 may be found in the teachings of WO 95/10616, WO 95/10618 and GB-B-2294048. These methods were applied to other species and subspecies to prepare the enzymes shown as sequences SEQ ID NO: 13-15, 17 and 19. Likewise, these teachings can be applied by the skilled artisan to isolate glucan lyases from other sources, preferably fungi, algae and bacteria (particularly cyanobacteria).

If the glucan contains links other than and in addition to the α-1,4- links the recombinant α-1,4-glucan lyase can be used in conjunction with a suitable reagent that can break the other links—such as a recombinant hydrolase—preferably a recombinant glucanohydrolase.

Glucosidases

As discussed above, α-1,4-glucan lyases specifically cleave the α-1,4-glucosidic bonds in starch or glycogen and convert glucosyl residues to anhydrofructose. α-glucosidases are members of the glycoside hydrolase family of enzymes, specifically family 31. Hydrolases also cleave the α-1,4-glucosidic bonds in starch or glycogen, and form glucose. Glycoside hydrolases and α-1,4-glucan lyases also share certain inhibitors, including analogs of products, substrates and transition state intermediates. Some examples include 1-deoxynojirimycin, acarbose, castanospermine, p-chloromercuribenzoic acid (PCMB) and bromoconduritol.

Interestingly, it has been found that α-1,4-glucan lyases and α-glucosidases share 23-28% sequence identity. A multiple sequence alignment further reveals that lyases and α-glucosidases contain seven well-conserved regions, three of which are recognized as the active site of family 31. The seven conserved regions are rich in charged and aromatic amino acid residues, which is a typical feature of enzymes involved in carbohydrate metabolism. (See Yu et al. 1999, Biochimica et Biophysica Acta 1433:1-15; Frandsen et al. 1998, Plant Mol. Biol. 37:1-13; Mori et al. 1999, 3^{rd} Carbohydrate Bioengineering Meeting, University of Newcastle Upon Tyne, Abstr. 7.7.)

Based on the shared substrate and inhibitor specificity, the type of bond cleaved, and the active site sequence similarity, the catalytic mechanism of α-1,4-glucan lyase is likely to resemble that of α-glucosidases. Therefore the use of α-glucosidases in the practice of the instant invention a contemplated embodiment.

Molecular Biology

General teachings of recombinant DNA techniques may be found in Sambrook, J., Fritsch, E. F., Maniatis T. (Editors) Molecular Cloning. A laboratory manual. Second edition. Cold Spring Harbour Laboratory Press. New York 1989.

In order to express a nucleotide sequence, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the gene may need to be suitably modified before transformation—such as by removal of introns.

Filamentous Fugni as Host Organisms

In one embodiment, the host organism can be of the genus Aspergillus, such as Aspergillus niger. A transgenic Aspergillus can be prepared by following the teachings of Rambosek, J. and Leach, J. 1987 (Recombinant DNA in filamentous fungi: Progress and Prospects. CRC Crit. Rev. Biotechnol. 6:357-393), Davis R. W. 1994 (Heterologous gene expression and protein secretion in Aspergillus. In: Martinelli S. D., Kinghorn J. R.(Editors) Aspergillus: 50 years on. Progress in industrial microbiology vol 29. Elsevier Amsterdam 1994. pp 525-560), Ballance, D. J. 1991 (Transformation systems for Filamentous Fungi and an Overview of Fungal Gene structure. In: Leong, S. A., Berka R. M. (Editors) Molecular Industrial Mycology. Systems and Applications for Filamentous Fungi. Marcel Dekker Inc. New York 1991, pp 1-29) and Turner G. 1994 (Vectors for genetic manipulation. In: Martinelli S. D., Kinghorn J. R.(Editors) Aspergillus: 50 years on. Progress in industrial microbiology vol 29. Elsevier Amsterdam 1994. pp. 641-666). However, the following commentary provides a summary of those teachings for producing transgenic Aspergillus.

For almost a century, filamentous fungi have been widely used in many types of industry for the production of organic compounds and enzymes. For example, traditional japanese koji and soy fermentations have used Aspergillus sp. Also, in this century Aspergillus niger has been used for production of organic acids particular citric acid and for production of various enzymes for use in industry.

There are two major reasons why filamentous fungi have been so widely used in industry. First filamentous fungi can produce high amounts of extracellular products, for example enzymes and organic compounds such as antibiotics or organic acids. Second filamentous fungi can grow on low cost substrates such as grains, bran, beet pulp etc. The same reasons have made filamentous fungi attractive organisms as hosts for heterologous expression of recombinant enzymes according to the present invention.

In order to prepare the transgenic Aspergillus, expression constructs are prepared by inserting a requisite nucleotide sequence into a construct designed for expression in filamentous fungi.

Several types of constructs used for heterologous expression have been developed. These constructs can contain a promoter which is active in fungi. Examples of promoters include a fungal promoter for a highly expressed extracellular enzyme, such as the glucoamylase promoter or the α-amylase promoter. The nucleotide sequence can be fused to a signal sequence which directs the protein encoded by the nucleotide sequence to be secreted. Usually a signal sequence of fungal origin is used. A terminator active in fungi ends the expression system.

Another type of expression system has been developed in fungi where the nucleotide sequence can be fused to a smaller or a larger part of a fungal gene encoding a stable protein. This can stabilize the protein encoded by the nucleotide sequence. In such a system a cleavage site, recognized by a specific protease, can be introduced between the fungal protein and the protein encoded by the nucleotide sequence, so the produced fusion protein can be cleaved at this position by the specific protease thus liberating the protein encoded by the nucleotide sequence. By way of example, one can introduce a site which is recognized by a KEX-2 like peptidase found in at least some Aspergilli. Such a fusion leads to cleavage in vivo resulting in protection of the expressed product and not a larger fusion protein.

Heterologous expression in Aspergillus has been reported for several genes coding for bacterial, fungal, vertebrate and plant proteins. The proteins can be deposited intracellularly if the nucleotide sequence is not fused to a signal sequence. Such proteins will accumulate in the cytoplasm and will usually not be glycosylated which can be an advantage for some bacterial proteins. If the nucleotide sequence is equipped with a signal sequence the protein will accumulate extracellularly.

With regard to product stability and host strain modifications, some heterologous proteins are not very stable when they are secreted into the culture fluid of fungi. Most fungi produce several extracellular proteases which degrade heterologous proteins. To avoid this problem special fungal strains with reduced protease production have been used as host for heterologous production.

For the transformation of filamentous fungi, several transformation protocols have been developed for many filamentous fungi (Ballance 1991, ibid). Many of them are based on preparation of protoplasts and introduction of DNA into the protoplasts using PEG and Ca.sup.2+ ions. The transformed protoplasts then regenerate and the transformed fungi are selected using various selective markers. Among the markers used for transformation are a number of auxotrophic markers such as argB, trpC, niaD and pyrG, antibiotic resistance markers such as benomyl resistance, hygromycin resistance and phleomycin resistance. A commonly used transformation marker is the amdS gene of A. nidulans, which, in high copy number, allows the fungus to grow with acrylamide as the sole nitrogen source.

Yeast as Host Organisms

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence into a construct designed for expression in yeast. Several types of constructs used for heterologous expression have been developed. The constructs contain a promoter active in yeast fused to the nucleotide sequence, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, e.g. G418.

Plants as Host Organisms

Another host organism is a plant. In this regard, the art is replete with references for preparing transgenic plants. Two documents that provide some background commentary on the types of techniques that may be employed to prepare transgenic plants are EP-B-0470145 and CA-A-2006454—some of which commentary is presented below.

The basic principle in the construction of genetically modified plants is to insert genetic information in the plant genome so as to obtain a stable maintenance of the inserted genetic material.

Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Thus, in one aspect, the present invention relates to a vector system which carries a recombinant nucleotide sequence and which is capable of introducing the nucleotide sequence into the genome of an organism, such as a plant, and wherein the nucleotide sequence is capable of preparing in situ an anti-oxidant.

The vector system may comprise one vector, but it can comprise at least two vectors. In the case of two vectors, the vector system is normally referred to as a binary vector system. Binary vector systems are described in further detail in Gynheung An et al. (1980), Binary Vectors, Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes (An et al. (1986), Plant Physiol. 81, 301-305 and Butcher D. N. et al. (1980), Tissue Culture Methods for Plant Pathologists, eds.: D. S. Ingrams and J. P. Helgeson, 203-208).

Several different Ti and Ri plasmids have been constructed which are suitable for the construction of the plant or plant cell constructs described above.

The nucleotide sequence of the present invention should preferably be inserted into the Ti-plasmid between the border sequences of the T-DNA or adjacent a T-DNA sequence so as to avoid disruption of the sequences immediately surrounding the T-DNA borders, as at least one of these regions appear to be essential for insertion of modified T-DNA into the plant genome.

As will be understood from the above explanation, if the organism is a plant, then the vector system of the present invention is preferably one which contains the sequences necessary to infect the plant (e.g. the vir region) and at least one border part of a T-DNA sequence, the border part being located on the same vector as the genetic construct. Preferably, the vector system is an Agrobacterium tumefaciens Ti-plasmid or an Agrobacterium rhizogenes Ri-plasmid or a derivative thereof, as these plasmids are well-known and widely employed in the construction of transgenic plants, many vector systems exist which are based on these plasmids or derivatives thereof.

In the construction of a transgenic plant the nucleotide sequence or construct or vector of the present invention may be first constructed in a microorganism in which the vector can replicate and which is easy to manipulate before insertion into the plant. An example of a useful microorganism is E. coli, but other microorganisms having the above properties may be used. When a vector of a vector system as defined above has been constructed in E. coli, it is transferred, if necessary, into a suitable Agrobacterium strain, e.g. Agrobacterium tumefaciens. The Ti-plasmid harbouring the first nucleotide sequence or construct of the invention is thus preferably transferred into a suitable Agrobacterium strain, e.g. A. tumefaciens, so as to obtain an Agrobacterium cell harbouring the promoter or nucleotide sequence or construct of the invention, which DNA is subsequently transferred into the plant cell to be modified.

As reported in CA-A-2006454, a large number of cloning vectors are available which contain a replication system in E. coli and a marker which allows a selection of the transformed cells. The vectors contain for example pBR322, the pUC series, the M13 mp series, pACYC 184 etc. In this way, the promoter or nucleotide or construct of the present invention can be introduced into a suitable restriction position in the vector. The contained plasmid is used for the transformation in E. coli. The E. coli cells are cultivated in a suitable nutrient medium and then harvested and lysed. The plasmid is then recovered and then analysed—such as by any one or more of the following techniques: sequence analysis, restriction analysis, electrophoresis and further biochemical-molecular biological methods. After each manipulation, the used DNA sequence can be restricted or selectively amplified by PCR techniques and connected with the next DNA sequence. Each sequence can be cloned in the same or different plasmid.

After each introduction method of the nucleotide sequence or construct or vector according to the present invention in the plants the presence and/or insertion of further DNA sequences may be necessary. If, for example, for the transformation the Ti- or Ri-plasmid of the plant cells is used, at least the right boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:1-46; and An et al., EMBO J. (1985) 4:277-284.

Direct infection of plant tissues by Agrobacterium is a simple technique which has been widely employed and which is described in Butcher D. N. et al. (1980), Tissue Culture Methods for Plant Pathologists, eds.: D. S. Ingrams and J. P. Helgeson, 203-208. For further teachings on this topic see Potrykus (Annu Rev Plant Physiol Plant Mol Biol [1991] 42:205-225) and Christou (Agro-Food-Industry Hi-Tech March/April 1994 17-27). With this technique, infection of a plant may be done on a certain part or tissue of the plant, i.e. on a part of a leaf, a root, a stem or another part of the plant.

Typically, with direct infection of plant tissues by Agrobacterium carrying the first nucleotide sequence or the construct, a plant to be infected is wounded, e.g. by cutting the plant with a razor or puncturing the plant with a needle or rubbing the plant with an abrasive. The wound is then inoculated with the Agrobacterium. The inoculated plant or plant part is then grown on a suitable culture medium.

When plant cells are constructed, these cells are grown and, optionally, maintained in a medium according to the present invention following well-known tissue culturing methods—such as by culturing the cells in a suitable culture medium supplied with the necessary growth factors such as amino acids, plant hormones, vitamins, etc, but wherein the culture medium comprises a component according to the present invention. Regeneration of the transformed cells into genetically modified plants may be accomplished using known methods for the regeneration of plants from cell or tissue cultures, for example by selecting the transformed shoots and by subculturing the shoots on a medium containing the appropriate nutrients, plant hormones, etc.

Further teachings on plant transformation may be found in EP-A-0449375.

Reference may even be made to Spngstad et al (1995 Plant Cell Tissue Organ Culture 40 pp 1-15) as these authors present a general overview on transgenic plant construction.

In one embodiment, the plant is a grapevine. There are a number of teachings in the art on how to prepare transformed grapevines. For example, reference may be made to Baribault et al (J Exp Bot 41 (229) 1990 1045-1050), Baribault et al (Plant Cell Rep 8 (3) 1989 137-140), Scorza et al (J Am Soc Horticultural Science 121 (4) 1996 616-619), Kikkert et al (Plant Cell Reports 15 (5) 1996 311-316), Golles et al (Acta Hortic 1997 vol 447 Number: Horticultural Biotechnology in vitro Culture and Breeding Pages 265-275), Gray and Scorza (WO-A-97/49277) and Simon Robinson et al (Conference abstracts and paper presented in Biotechnology—Food and Health for the 21st Century, Adelaide, Australia, 1998). By way of example Robinson et al (ibid) disclose a method for transforming grapevine wherein somatic embryos are induced on callus formed from another tissue and Agrobacterium infection is used to transfer target genes into the embryo tissue.

Further reference may be made to the teachings of Andrew Walker in Nature Biotechnology (Vol 14, May 1996, page 582) who states that:
“The grape, one of the most important fruit plants in the world, has been difficult to engineer because of its high levels of tannins and phenols, which interfere with cell culture and transformation; the compounds oxidize quickly and promote the decay of grape cells.”

In that same edition of Nature Biotechnology, Perl et al (pages 624-628) report on the use of the combination of polyvinylpolypyrrolidone and dithiothreitol to improve the viability of grape transformation during Agrobacterium infection.

Hence, the present invention provides an alternative means for transforming grape. In this regard, the antioxidant that is prepared in situ by a grape transformed in accordance with the present invention improves the viability of grape transformation during Agrobacterium infection.

Thus, according to one aspect of the present invention, there is provided the use of an antioxidant prepared in situ in order to effectively transform a grape.

In some instances, it is desirable for the recombinant enzyme or protein to be easily secreted into the medium to act as or to generate an anti-oxidant therein. In such cases, the DNA encoding the recombinant enzyme is fused to inter alia an appropriate signal sequence, an appropriate promoter and an appropriate terminator from the chosen host.

For example, for expression in Aspergillus niger the gpdA (from the Glyceraldehyde-3-phosphate dehydrogenase gene of Aspergillus nidulans) promoter and signal sequence is fused to the 5′ end of the DNA encoding the mature lyase. The terminator sequence from the A. niger trpC gene is placed 3′ to the gene (Punt, P. J. et al 1991—(1991): J. Biotech. 17, 19-34). This construction is inserted into a vector containing a replication origin and selection origin for E. coli and a selection marker for A. niger. Examples of selection markers for A. niger are the amdS gene, the argB gene, the pyrG gene, the hygB gene, the BmlR gene which all have been used for selection of transformants. This plasmid can be transformed into A. niger and the mature lyase can be recovered from the culture medium of the transformants. Eventually the construction could be transformed into a protease deficient strain to reduce the proteolytic degradation of the lyase in the medium (Archer D. B. et al 1992—Biotechnol. Lett. 14, 357-362).

In addition, and as indicated above, aside from using Aspergillus niger as the host, there are other industrial important microorganisms which could be used as expression systems. Examples of these other hosts include: Aspergillus oryzae, Aspergillus sp., Trichoderma sp., Saccharomyces cerevisiae, Kluyveromyces sp., Hansenula sp., Pichia sp., Bacillus subtilis, B. amyloliquefaciens, Bacillus sp., Streptomyces sp. or E. coli.

In accordance with the present invention, a suitable marker or selection means may be introduced into the host that is to be transformed with the nucleotide sequence. Examples of suitable markers or selection means are described in any one of WO-A-93/05163, WO-A-94/20627, GB patent application No. 9702591.0 (filed Feb. 7, 1997), GB patent application No. 9702576.1 (filed Feb. 7, 1997), GB patent application No. 9702539.9 (filed Feb. 7, 1997), GB patent application No. 9702510.0 (filed Feb. 7, 1997) and GB patent application No. 9702592.8 (filed Feb. 7, 1997).

In summation, the present invention relates to a process comprising preparing a medium that comprises an anti-oxidant and at least one other component, the process comprising preparing in situ in the medium the anti-oxidant; and wherein the anti-oxidant is prepared from a glucan by use of recombinant DNA techniques and/or the anti-oxidant is prepared by use of a recombinant glucan lyase. In a particularly preferred embodiment, the anti-oxidant is anhydro-fructose.

Various preferred features and embodiments of the present invention will now be described in more detail by way of non-limiting examples.

### EXAMPLES

Transgenic Potato

General teachings on potato transformation may be found in our co-pending patent applications PCT/EP96/03053, PCT/EP96/03052 and PCT/EP94/01082 (the contents of each of which are incorporated herein by reference).

For the present studies, the following protocol is adopted.

Plasmid Construction

To target glucan lyase protein accumulation to potato plastids, a DNA fragment with gene bank accession number Y18737, corresponding to the protein sequence Seq ID No 1 from W098/50532, called GLq1, was inserted into the cloning vector pBluescript KS⁺ from Stratagene as described in International Patent Application Number W095/10618A3.

From this DNA fragment, the first 49 amino acids were exchanged with a DNA fragment containing the first 55 amino acids of the ribulose bisphosphate carboxylase small chain lb precursor, which encodes an efficient plastid transit peptide by overlapping PCR. First, the plastid transit peptide of ribulose bisphosphate carboxylase small chain 1b precursor (Dedonder et al. (1993), Plant Physiol. 101: 801-8) was amplified by PCR primers:
5′-TGCTCTAGAGAACAATGGCTTCCTCTATGC-3′ (SEQ ID NO: 23) and 5′-GTTTGTCGGACAATGCGGTCATGCAGTTAACTCTTCCGCC-3′ (SEQ ID NO: 24).

Secondly, a 413 bp DNA fragment of the 5′ end of GLq1 was amplified by PCR primers:
5′-TGCTCTAGACAACAATGTTTTCAACCCTTGCGTTTGTC-3′ (SEQ ID NO: 25) and
5′-GTATGACGTGACCTGAACCTG-3′ (SEQ ID NO: 26).

The Two DNA fragments was mixed together in equal amounts, heated to 95 C for 3 min and renatured. PCR amplification with primers:
5′-TGCTCTAGAGAACAATGGCTTCCTCTATGC-3′ (SEQ ID NO: 27) and
5′-GTATGACGTGACCTGAACCTG-3′ (SEQ ID NO: 28) resulted in a DNA fragment where the plastid transit peptide from ribulose bisphosphate carboxylase small chain 1b precursor was fused to the 5′ end of the GLq1 DNA fragment. This fragment was digested with XbaI and SmaI and reinserted into the GLq1 gene in the cloning vector pBluescript KS⁺ to generate the modified GLq1 gene designated signal-GLq1.

The complete coding sequence of signal-GLq1 was digested with XbaI and BglII and inserted between the 35S promoter and the E9 terminator sequence in plasmid pCAMBIA 2300-35S-E9 digested with the same enzymes to create the final plasmid pCAMBIA 2300-35S-signal-GLq1-E9.

The disarmed Agrobacterium tumefaciens strain LBA 4404, containing the helper vir plasmid pRAL4404 (Hoekema et al, 1983 Nature 303 pp 179-180), was cultured on YMB agar (K₂HPO₄.3H₂O 660 mg 1⁻¹, MgSO₄ 200 mg 1⁻¹, NaCl 100 mg 1⁻¹, mannitol 10 g 1⁻¹ yeast extract 400 mg 1⁻¹, 0.8% w/v agar, pH 7.0) containing 100 mg 1⁻¹ rifampicin 50 mg 1⁻¹ gentamycin sulphate. Transformation with pCAMBIA 2300-35S-signal-GLq1-E9 was accomplished using the freeze-thaw method of Holters et al (1978 Mol Gen Genet 163 181-187) and transformants were selected on YMB agar containing 100 mg 1⁻¹ rifampicin and 500 mg 1⁻¹ kanamycin, and 50 mg 1⁻¹ gentamycin sulphate.

Transformation of Plants

Shoot cultures of Solanum tuberosum cv Saturna were maintained on LS agar containing Murashige Skoog basal salts (Sigma M6899) (Murashige and Skoog, 1965, Physiol Plant 15 473-497) with 2 μM silver thiosulphate, and nutrients and vitamins as described by Linsmaier and Skoog (1965 Physiol Plant 18 100-127). Cultures were maintained at 25° C. with a 16 h daily photoperiod. After approximately 40 days, subculturing was performed during which leaves were removed, and the shoots were cut into mononodal segments of approximately 8 mm length.

Shoot cultures of approximately 40 days maturity (5-6 cm height) were cut into 8 mm internodal segments which were placed into liquid LS-medium containing Agrobacterium tumefaciens transformed with pCAMBIA 2300-35S-signal-GLq1-E9. Following incubation at room temperature for 30 minutes, the segments were dried by blotting onto sterile filter paper and transferred to LS agar (0.8% w/v containing 2 mg 1⁻¹ 2,4-D and 500 μg 1⁻¹ trans-zeatin. The explants were covered with filter paper, moistened with LS medium, and covered with a cloth for three days at 25° C. Following this treatment, the segments were washed with liquid LS medium containing 800 mg 1⁻¹ carbenicillin, and transferred on to LS agar (0.8% w/v) containing 1 mg 1⁻¹ trans-zeatin, 100 μg 1⁻¹ gibberellic acid (GA3), with sucrose (e.g. 7.5 g 1⁻¹) and glucosamine (e.g. 2.5 g 1⁻¹) and 500 mg 1⁻¹ kanamycin as the selection agent.

The segments were sub-cultured to fresh substrate each 3-4 weeks. In 3 to 4 weeks, shoots develop from the segments and the formation of new shoots continues for 3-4 months.

Rooting of Regenerated Shoots

The regenerated shoots were transferred to rooting substrate composed of LS-substrate, agar (8 g/l) and carbenicillin (800 mg/l).

The transgenic genotype of the regenerated shoots was verified by PCR. A leaf was excised from plants showing normal root growth on medium containing kanamycin. DNA was extracted according to Dellaporta et. Al., 1983, Plant Mol. Biol. Rep. 1, 19-21. PCR amplification using primers:
5′-AGCGGATAACAATTTCACACAGGA-3′ (SEQ ID NO: 29) and
5′-GTATGACGTGACCTGAACCTG-3′ (SEQ ID NO: 30), specific for the inserted T-DNA revealed that all plants showing normal root growth on medium containing kanamycin produced a band of the right size whereas in untransformed control plants no band was detected.

In total, 24 individual transgenic plants were produced that showed resistance towards kanamycin and contained the glucan lyase gene.

Transgenic plants may also be verified by performing a GUS assay on the co-introduced α-glucuronidase gene according to Hodal, L. et al. (Pl. Sci. (1992), 87:115-122).

Alternatively, the transgenic genotype of the regenerated shoot may be verified by performing NPTII assays (Radke, S. E. et al, Theor. Appl. Genet. (1988), 75: 685-694) or by performing PCR analysis according to Wang et al (1993, NAR 21 pp 4153-4154).

Glucan Lyase Activity in Plants

To determine whether these 24 transgenic lines expressed and accumulated a functional glucan lyase gene, total protein extracts was prepared from leaves of wild type and transgenic potato plants. Approximately 200 mg of leaf tissue was ground in 0.6 ml protein extraction buffer (20 mM Tris pH 7,5, 150 mM NaCl and 1 mM EDTA) and centrifuged for 20 minutes at 15,000 rpm at 4° C. The supernatant was transferred to a new tube at kept at 4 deg C. The protein concentration was determined by a Bio-Rad Protein Assay (Bio-Rad Laboratories) according to the recommendations of the manufacturer. Typically, 5 mg/ml of protein was extracted by this procedure. To detect glucan lyase activity, 0.5 mg total protein extract was diluted with 100 mM potassium acetate pH 5.5 to 0.250 ml. Then 0.250 ml 100 mM potassium acetate, pH 5.5, with 10 mg/ml glycogen, was added and the reaction was incubated at 40° C. for 60 minutes. The reaction was stopped by heating at 100° C. for 2 minutes before anhydrofructose was detected by 3.5 dinitrosalicylic acid under alkaline conditions, as described by Yu et. al in W094/09122.

Results showed that, of the 24 individual transgenic potato lines, two lines did not produce detectable anhydrofructose, seven lines produced less anhydrofructose than 50 micrograms per 0.5 mg protein extract. The resulting 15 lines produced from 165 to 524 micrograms anhydrofructose (AF) per 0.5 mg total protein extract (Table 4).

TABLE 4

| | | Glucan Lyase activity | |
|---|---|---|---|
| | | microgram AF/ | Lines tested for |
| | Trangenic as | 500 microgram | anhydrofructose |
| Line No. | tested by PCR | protein | in situ |
| 5.1 | yes | 470.3 | |
| 6.1 | yes | 263.8 | |
| 8.1 | yes | 0 | yes |
| 9.1 | yes | 247.3 | |
| 11.1 | yes | 409.1 | |
| 14.1 | yes | 210.8 | yes |
| 14.2 | yes | 0 | |
| 14.3 | yes | 337.2 | yes |
| 11.2 | yes | 524 | |
| 22 | yes | 234.8 | |
| 22.2 | yes | 427.4 | |
| 23.2 | yes | 0 | |
| 24 | yes | 511.1 | |
| 21.3 | yes | 48.6 | |
| 23.4 | yes | 36.5 | |
| 17.2 | yes | 28 | |
| 33.1 | yes | 0 | |
| 27 | yes | 164.9 | |
| 31 | yes | 510.1 | |
| 29 | yes | 456.1 | yes |
| 6.2 | yes | 23.0 | |
| 6.3 | yes | 23 | |
| 4.4 | yes | 0 | |
| 31.1 | yes | 432.4 | |
| wt | no | 0 | yes |

Regular visual inspection of the plants did not reveal phenotypic alterations between the 24 transgenic lines or when compared to wild type plants grown under the same conditions without kanmycin in the growth medium. These results demonstrate that plants can be engineered to express an active glucan lyase gene and cultivated on synthetic medium under sterile conditions without negative effects on their growth.

Detection of Anhydrofructose in Transgenic Plants

Since potato plants grown on synthetic LS medium only synthesize very low amounts of starch, transgenic and wild type plants (height approx. 8-10 cms) were transferred to soil and placed in a growth chamber. When growth was well established, the plants were transferred to a greenhouse and grown for four weeks.

Glucan lyase activity was re-determined as described above and no significant differences in protein activity were observed.

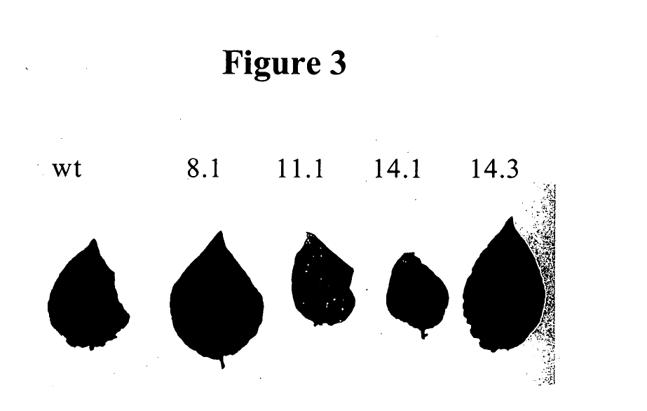

Glucan lyases produce anhydrofructose from starch. To analyse for differences in the starch content between soil-grown wild type plants and transgenic plants accumulating active glucan lyase, leaves were stained with iodine according to Visser et al., 1991 Mol. Gen. Genet. 225, 289-296. Leaves from plants grown in the greenhouse with a 16-hour photoperiod were excised from the plants after they had received 8-hour of light. Qualitative starch determination clearly shows that wild type plants and the transgenic line 8.1 (in which no active glucan lyase could be detected) contain high levels of starch. In contrast, no or very little starch could be detected from transgenic lines 11.1, 14.1 and 14.4. These results suggest that the introduced glucan lyase degrades the starch for the production of anhydrofructose (FIG. 3).

To determine anhydrofructose accumulation in the transgenic plants, neutral and phosphorylated sugars were extracted. Approximately 300 mg of leaf tissue was transferred to 50 ml polypropylene falcon tubes and frozen in liquid nitrogen. After evaporation of the liquid nitrogen, 5 ml of 80% EtOH was added and the tubes were placed in a water bath at 80° C. for 15 minutes. The supernatant was removed to new tubes and the plant material was re-extracted with 5 ml 25% EtOH at 0° C. for 30 minutes, following a re-extraction with 2 ml water at 0° C. for 15 minutes. The plant material was washed twice with 2 ml water and all the supernatants were combined. 2 ml of dichlormethane was added and the tubes were mixed gently before centrifugation for 10 minutes at 3000 rpm. The water phase was transferred to new tubes and the dichlormethane extraction was repeated. The water was evaporated by freeze-drying overnight and the final pellet was dissolved in 200 μl H₂O.

The presence of anhydrofructose was determined by reacting 200 μl total sugar extract with 200 μl 3,5 dinitrosalicylic acid for 10 minutes at room temperature before the absorbance of the reaction mixture at 546 nm was determined, as described by Yu et al. in WO94/09122. The results are shown in Table 5.

TABLE 5

| | | | | | |
|---|---|---|---|---|---|
| | Reaction | | Absorbance | Micrograms | μg AF/g fresh |
| Sample | Temp. (° C.) | Reaction Time | 546 nm | AF | weight |

Before dephosphorylation

| | | | | | |
|---|---|---|---|---|---|
| Blank | 40 | 10 min | 0 | | |
| Wt | 40 | 10 min | 0.003 | 0 | 0 |
| line 8.1 | 40 | 10 min | 0.005 | 0 | 0 |
| line 11.1 | 40 | 10 min | 0.017 | 2.2 | 7.1 |
| line 14.1 | 40 | 10 min | 0.021 | 2.8 | 9.2 |
| line 14.3 | 40 | 10 min | 0.023 | 3.1 | 10.3 |
| line 29 | 40 | 10 min | 0.019 | 2.5 | 8.2 |

After dephosphorylation

| | | | | | |
|---|---|---|---|---|---|
| Blank | 40 | 10 min | 0 | | |
| wt | 40 | 10 min | 0.009 | 0 | 0 |
| line 8.1 | 40 | 10 min | 0.007 | 0 | 0 |
| line 11.1 | 40 | 10 min | 0.041 | 12.3 | 37.9 |
| line 14.1 | 40 | 10 min | 0.048 | 14.6 | 45.0 |
| line 14.3 | 40 | 10 min | 0.039 | 11.9 | 34.9 |
| line 29 | 40 | 10 min | 0.033 | 9.5 | 28.1 |

The results show that anhydrofructose could neither be detected in wild type potato leaves nor in line 8.1, which does not accumulate detectable glucan lyase activity. However, in lines 11.1, 14.1 14.3 and 29, 9-12 μg anhydrofructose/g fresh weight were detected.

Many sugars also exist in phorphorylated forms. It has been shown that yeast and rat brain hexokinases phosphorylate 1,5-anhydro-D-fructose and its metabolite, anhydroglucitol (Taguchi et al., 1993, Biotechnol. Appl. Biochem. 18, 275-83). Therefore, neutral and phosphorylated sugars were incubated in the presence of calf intestinal alkaline phosphatase (CIAP, Roche) to dephosphorylate all phosphorylated sugars that are substrates for the phosphatase. Total sugars were re-extracted from 600 mg leaf tissue and dissolved in 200 μl H₂O as described above. 100 μl sugar extract was incubated with 50 units CIAP in a reaction volume of 200 μl at 37° C. for 4 hrs, in the buffer recommended by the manufacture, before anhydrofructose was measured as described above. Between 37 and 54 μg anhydrofructose/g fresh weight were detected in the dephosphorylated samples of lines 11.1, 14.1 14.3 and 29, demonstrating that transgenic potato plants engineered to accumulate active glucan lyase produce anhydrofructose.

A more sensitive method to detect anhydrofructose, described by Kametani et al. (1996 J. Biochem. 119, 180-185), was used to confirm the above results.

Total sugar extracts were isolated from wild type plants and transgenic line 11.1. One gram of leaf tissue was homogenized using a Kontes grinder no.21 in 5 ml 150 mM NaCl, followed by the addition of 6 ml 40 mM Tris HCl pH 6.0 and 35 mg O-ethylhydroxylamine. Cell debris was removed by centrifugation at 5000×g for 15 minutes. The supernatants were transferred to new tubes and left at room temperature in the dark for 12 hours for the derivatization of anhydrofructose. The samples were applied onto a reverse-phase column mounted on a computer aided HPLC system. The column was developed with a linear gradient from 100% water to acetonitrile/water (1:1 by volume) in 30 minutes at a flow rate of 1 ml/min and the elution was monitored by UV absorbance at 207 nm.

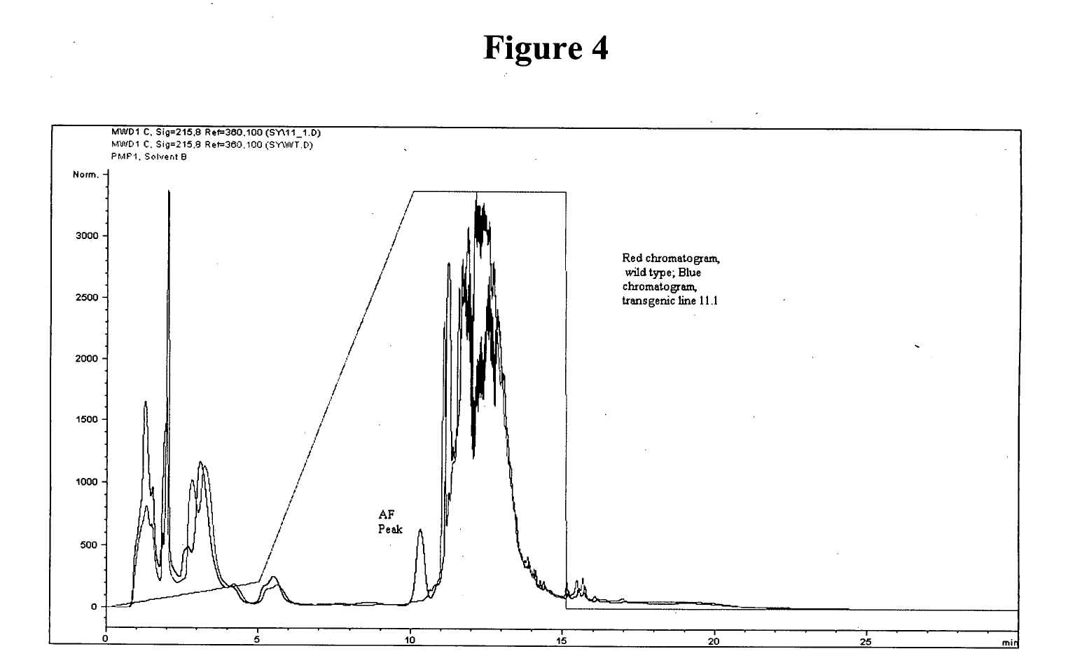

As shown in FIG. 4, anhydrofructose, eluting with a retention time of approximately 10 minutes, could clearly be detected from extracts of transgenic line 11.1, whereas anhydrofructose was absent in extracts of wild type plants. Quantitative determination of anhydrofructose in extracts analyzed by reverse phase HPLC corresponded well with results obtained from the assays using 3,5 dinitrosalicylic acid.

Transgenic Arabidopsis

Construction of plasmid pCAMBIA 2300-35S-signal-GLq1-E9 is described above. Transformation of Arabidopsis thaliana plants was performed as described above for potato plants.

Glucan Lyase Activity in Arabidopsis Plants

To determine whether transgenic Arabidopsis accumulates a functional glucan lyase gene, total protein extracts were prepared from leaves of wild type and transgenic lines SR1 and SR2. Approximately 100 mg of leaf tissue was ground up in 0.3 ml protein extraction buffer (20 mM Tris pH 7.5, 150 mM NaCl and 1 mM EDTA) and centrifuged for 20 minutes at 15,000 rpm at 4° C. The supernatant was transferred to a new tube at kept at 4° C. Protein concentration was determined by a Bio-Rad Protein Assay (Bio-Rad Laboratories), according to the recommendations of the manufacturer. To detect glucan lyase activity, 250 μg total protein extract was diluted with 100 mM potassium acetate, pH 5.5, to 250 μl. Then 250 μl of 100 mM potassium acetate, pH 5.5, with 10 mg/ml glycogen, was added and the reaction mixture was incubated at 40° C. for 60 minutes. The reaction was stopped by heating at 100° C. for 2 minutes before anhydrofructose was determined by 3,5 dinitrosalicylic acid under alkaline conditions as described by Yu et al. in WO94/09122.

Results showed that transgenic lines SR1 and SR2 produced 82-136 μg anhydrofructose per 250 μg total protein extract. No anhydrofructose was detected in extracts from wild type plants.

These results demonstrate that Arabidopsis thaliana can be engineered to express glucan lyase and accumulate the product of its activity.

To analyse for differences in the starch content between soil-grown wild type plants and transgenic plants accumulating active glucan lyase, leaves were stained with iodine by the same procedure as used for potato.

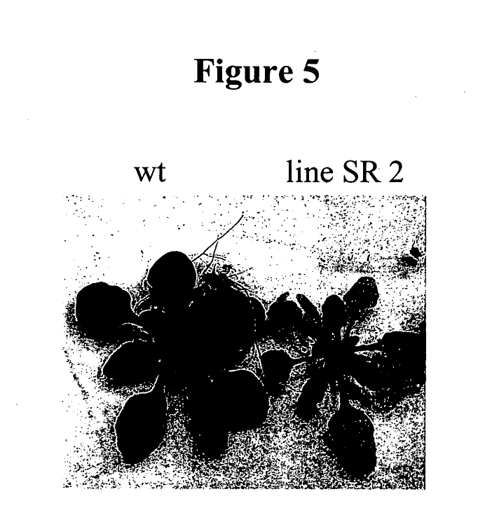

Qualitative starch determination clearly showed that wild type plants contain high levels of starch. In contrast, no or very little starch could be detected in the transgenic line SR2. These results show that the introduced glucan lyase degrades starch. As the only product of the degradation of starch by a glucan lyase is anhydrofructose, the data confirm the production of anhydro fructose in transgenic Arabidopsis plants expressing a glucan lyase (FIG. 5).

Transgenic Grape

Transformed grapes are prepared following the teachings of Perl et al (ibid) but wherein the use of the combination of polyvinylpolypyrrolidone and dithiothreitol is optional. In these studies, the grapes are transformed with any one of the nucleotide sequences presented as SEQ ID NO: 7-12, 16, 18 or 20. The transformation leads to in situ preparation of 1,5-anhydro-D-fructose. The transformed grapes are beneficial for one or more of the reasons mentioned earlier.

Details on these studies are as follows.

Tissue-Culture Systems for Transformation Studies

The long term somatic embryogenic callus culture is developed from the vegetative tissues of anthers of Vitis vinifera CV Superior Seedless. Methods for another culture, induction of somatic embryogenesis and maintenance of embryogenic cultures, are previously described (Perl et al, 1995, Plant Sci 104: 193-200). Briefly, embryogenic calli are maintained on solidified (0.25% gelrite) MS medium (Murashige and Skoog, 1962, Physiol Plant 15: 473-497) supplemented with 6% sucrose, 2 mg/L 2,4-diclorophenoxyacetic acid (2,4-D), 5 mg/L Indole-3-aspartic acid (IASP), 0.2 mg/L 6-benzyladenine (BAP) and 1 mg/L abscisic acid (ABA). Proembryogenic calli are induced by transferring the calli to MS medium supplemented with the same phytohormones, but 2,4-D is substituted with 2 mg/L 2-naphthoxyacetic acid (NOA). This stage is used for transformation experiments.

Agrobacterium Strains

For studying the sensitivity of grape embryogenic calli to the presence of different Agrobacterium strains, or for stable transformation experiments, cocultivation is attempted using the following A. tumefaciens strains: EHA 101-p492 (Perl et al. 1993, Bio/Technology 11:715-718); LBA 4404-pGPTV (Becker et al, 1992, Plant Mol Biol 20: 1195-1197); and GVE 3101-pPCV91 (Vancanneyt et al, 1990, Mol Gen Genet 220: 245-250). These strains contain the binary vectors conferring resistance to kanamycin (nptII), basta (bar) and hygromycin (hpt), respectively, all under the control of the nopalin-synthase (NOS) promoter and terminator. Bacteria are cultured with the proper antibiotics in liquid LB medium for 24 hours at 28° C. at 200 rpm.

Cocultivation

For studying the sensitivity of grape embryogenic calli to different Agrobacterium strains, bacterial cultures with different optical densities (0.1-0.7 at 630 nm) are prepared from an overnight culture of Agrobacterium strains. Bacteria are centrifuged 5 minutes, 5000 rpm and resuspended in antibiotic free McCown's Woody Plant Medium (WPM) (Lloyd and McCown, 1981, Int Plant Prop Soc Proc 30: 421-427). Three grams fresh weight of embryogenic calli (7 days after transfer to NOA containing medium) are resuspended in 10 ml of overnight cultured bacterial suspensions for 5 minutes, dry blotted and transferred to Petri dishes containing regeneration medium [basal WPM medium supplemented with thidiazuron (TDZ) (0.5 mg/L), Zeatin riboside (ZR) (0.5 mg/L), and sucrose (3%)]. The regeneration medium is solidified with gelrite (0.25% w/v) and the calli, after initial drainage of excess bacteria, are cocultivated in the dark at 25° C. for different times (5 minutes up to 7 days). For stable transformation experiments, inoculum (OD 0.6 at 630 nm) is prepared from an overnight culture of LBA 4404 or GVE 3101. Bacteria are centrifuged 5 minutes, 5000 rpm and resuspended in antibiotic-free WPM medium. Embryogenic calli (3 g fresh weight) are resuspended in 10 ml of bacteria for 5 minutes, dry blotted and transferred to Petri dishes containing solidified (0.25% w/v) gelrite regeneration medium supplemented with different antioxidants. The calli are cocultivated for 48 hours in the dark at 25° C.

Selective Culture

Following 48 hours of cocultivation, the embryogenic callus is maintained in the dark for 7 days on antioxidant containing regeneration medium. Subsequently, the calli are collected on a sterile metal screen and transferred to fresh WPM regeneration medium at 25° C. under 40 μE/m.sup.2/s (white fluorescent tubes). All regeneration media are supplemented with 400 mg/L claforan, 1.5 g/L malt extract and different selectable markers: kanamycin (50-500 mg/L), hygromycin (15 mg/L) and Basta (1-10 mg/L). Periodic increases in hygromycin concentration are used. The putative transformed calli are cultured on regeneration medium supplemented with 15 mg/L hygromycin. Every two weeks the regenerating calli are transferred to fresh medium supplemented with 20 and 25 mg/L hygromycin respectively. Control, untransformed grape calli are also cultured on selective media and are periodically exposed to increasing hygromycin concentrations. Green adventitious embryos, which developed on calli cultured for 8-10 weeks on selective regeneration medium, are transferred to germination medium. Embryo germination, rooting and subsequent plantlet development are induced on WPM as described (Perl et al, 1995, Plant Sci 104: 193-200), supplemented with 25 mg/L hygromycin or 10 mg/L basta. Conversion of vitrified abnormal plantlets into normal-looking grape plantlets are obtained using solidified WPM medium supplemented with 0.1 mg/L NAA as described (Perl et al, 1995, Plant Sci 104: 193-200).

Transgenic Maize Plants

Introduction

Since the first publication of production of transgenic plants in 1983 (Leemans, 1993 Biotechnology 11 s22), there have been numerous publications of production of transgenic plants including especially dicotyledon crop plants.

Until very recently there are very few reports on successful production of transgenic monocotyledononary crop plants. This relatively slow development within monocots are due to two causes. Firstly, until the early 1980s, efficient regeneration of plants from cultured cells and tissues of monocots had proven very difficult. This problem is ultimately solved by the culture of explants from immature and embryogenic tissue, which retain their morphogenic potential on nutrient media containing plant growth regulators. Secondly, the monocots are not a natural host for Agrobacterium tumefaciens, meaning that the successful developed techniques within the dicots using their natural vector Agrobacterium tumefaciens is unsuccessful for many years in the monocots.

Nevertheless, it is now possible to successfully transformation and produce fertile transgenic plants of maize using methods such as: (1) Silicon Carbide Whiskers; (2) Particle Bombardment; (3) DNA Uptake by PEG treated protoplast; or (4) DNA Uptake in Electroporation of Tissue. Each of these methods—which are reviewed by Thompson (1995 Euphtytica 85 pp 75-80)—may be used to prepare inter alia transgenic maize according to the present invention.

In particular, the Particle Gun method has been successfully used for the transformation of monocots. However, EP-A-0604662 reports on a different method of transforming monocotyledons. The method comprises transforming cultured tissues of a monocotyledon under or after dedifferentiation with Agrobacterium containing a super binary vector as a selectable marker a hygromycin-resistant gene is used. Production of transgenic calli and plant is demonstrated using the hygromycin selection. This method may be used to prepare inter alia transgenic maize according to the present invention.

Subsequent to the method of EP-A-0604662, EP-A-0672752 reports on non-dedifferentiated immature embryos. In this regard, both hygromycin-resistance and PPT-resistance genes are used as the selectable marker, with PPT giving rise to 10% or more independent transformed plants. This method may be used to prepare inter alia transgenic maize according to the present invention.

To date, it would appear that transgenic maize plants can be successfully produced from easily-culturable varieties—such as the inbred line A188. In this regard, see the teachings of Ishida et al (1996 Nature Biotechnology 14 pp 745-750). The method disclosed by these workers may be used to prepare inter alia transgenic maize according to the present invention.

Vasil (1996 Nature Biotechnology 14 pp 702-703) presents a further review article on transformation of maize. Even though it is possible to prepare transformed maize by use of, for example, particle Gun mediated transformation, for the present studies the following protocol is adopted.

Plasmid Construction

The disarmed Agrobacterium tumefaciens strain LBA 4404, containing the helper vir plasmid pRAL4404 (Hoekema et al, 1983 Nature 303 pp 179-180), is cultured on YMB agar (K₂HPO₄.3H₂O 660 mg 1⁻¹, MgSO₄ 200 mg 1⁻¹, NaCl 100 mg 1⁻¹, mannitol 10 g 1⁻¹, yeast extract 400 mg 1⁻¹, 0.8% w/v agar, pH 7.0) containing 100 mg 1⁻¹ rifampicin and 500 mg 1⁻¹ streptomycin sulphate. Transformation with pVICTOR IV GNG E35S nagB IV2′ or pVICTOR IV GNG rbc nagB IV2′ or pVICTOR IV GNG E35S nagB′ is accomplished using the freeze-thaw method of Holters et al (1978 Mol Gen Genet 163 181-187) and transformants are selected on YMB agar containing 100 mg 1⁻¹ rifampicin and 500 mg 1⁻¹ streptomycin, and 50 mg 1⁻¹ gentamycin sulphate.

Isolation and Cocultivation of Explants

Immature embryos of, for example, maize line A188 of the size between 1.5 to 2.5 mm are isolated and cocultivated with Agrobacterium tumefaciens strain LBA 4404 in N6-AS for 2-3 days at 25° C. under illumination. Thereafter, the embryos are washed with sterilized water containing 250 mg/l of cefotaxime and transferred to an LS medium and 250 mg/l cefotaxime and glucosamine in concentrations of up to 100 mg/l (the medium is hereafter called LSS1).

Conditions for the Selection of Transgenic Plants

The explants are cultured for three weeks on LSS1 medium and then transferred to an LS medium containing glucosamine and cefotaxime. After three weeks on this medium, green shoots are isolated.

Rooting of Transformed Shoots

Transformed shoots are transferred to an MS medium containing 2 mg/l for rooting. After four weeks on this medium, plantlets are transferred to pots with sterile soil for acclimatisation.

Transgenic Guar Plants

Transformation of guar cotyledonary explants is performed according to Joersbo and Okkels (PCT/DK95/00221) using Agrobacterium tumefaciens LBA4404 harbouring a suitable plasmid.

Other plants may be transformed in accordance with the present invention, such as other fruits, other vegetables, and other plants such as coffee plants, tea plants etc.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A method for increasing anhydrofructose levels in a plant or part thereof, the method comprising introducing a nucleic acid encoding glucan lyase into the plant or part thereof, wherein the glucan lyase is expressed and acts on a glucan substrate present in the plant or part thereof to yield increased levels of anhydrofructose in the plant or part thereof.

2. The method according to claim 1, wherein the glucan comprises α-1,4 links.

3. The method according to claim 2, wherein the glucan is starch.

4. The method according to claim 1, wherein the enzyme is an α-1,4-glucan lyase.

5. The method according to claim 4, wherein the glucan lyase comprises an amino acid sequence having at least 75% sequence identity to SEQ ID NO:1, SEQ ID NO:2, SEQ ID NO:3, SEQ ID NO:4, SEQ ID NO:5, SEQ ID NO:6, SEQ ID NO:13, SEQ ID NO:14, SEQ ID NO:15, SEQ ID NO:17 or SEQ ID NO:19.

6. The method according to claim 1, wherein the anhydrofructose is 1,5-D-anydrofructose.

7. The method according to claim 1, wherein the anhydrofructose is produced in the plant, or part thereof, and is then released into a medium.

8. The method according to claim 7, wherein the medium is, or is used in the preparation of, a foodstuff.

9. The method according to claim 8, wherein the foodstuff is a beverage.

10. The method according to claim 9, wherein the beverage is an alcoholic beverage.

11. The method according to claim 9, wherein the beverage is wine.

12. The method according to claim 1, wherein the plant or part thereof is all or part of a cereal or a fruit.

13. The method according to claim 1, wherein the plant is grape.

14. The method according to claim 1, wherein the plant is potato.

15. A method for improving stress tolerance in a plant comprising introducing a nucleic acid encoding glucan lyase into the plant, wherein the glucan lyase is expressed and acts on a glucan substrate present in the plant or part thereof to yield increased levels of anhydrofructose in the plant, thereby improving stress tolerance in the plant.

16. The method according to claim 15, wherein the glucan lyase comprises an amino acid sequence having at least 75% sequence identity to SEQ ID NO:1, SEQ ID NO:2, SEQ ID NO:3, SEQ ID NO:4, SEQ ID NO:5, SEQ ID NO:6, SEQ ID NO:13, SEQ ID NO:14, SEQ ID NO:15, SEQ ID NO:17 or SEQ ID NO:19.

17. A method for improving the transformation of a grape plant, comprising introducing a nucleic acid encoding glucan lyase into the plant, wherein the glucan lyase is expressed and acts on a glucan substrate present in the plant to yield increased levels of anhydrofructose in the plant, thereby improving transformation of the plant.

18. The method according to claim 17, wherein the glucan lyase comprises an amino acid sequence having at least 75% sequence identity to SEQ ID NO:1, SEQ ID NO:2, SEQ ID NO:3, SEQ ID NO:4, SEQ ID NO:5, SEQ ID NO:6, SEQ ID NO:13, SEQ ID NO:14, SEQ ID NO:15, SEQ ID NO:17 or SEQ ID NO:19.

19. A method for increasing the degradation of an α-1,4-glucan substrate in a plant or part thereof, the method comprising introducing a nucleic acid encoding an enzyme selected from the group consisting of α-glucosidase and α-1,4-glucan lyase into the plant or part thereof, wherein the enzyme is expressed and acts on the glucan substrate to yield increased degradation of the glucan substrate in the plant or part thereof.

20. The method according to claim 19, wherein the α-1,4-glucan substrate is starch.

21. The method according to claim 19, wherein the α-1,4-glucan substrate is glycogen.

22. The method according to claim 19, wherein the α-1,4-glucan substrate is maltose, a maltosaccharide, a polymer thereof, or a combination thereof.
